(19) United States
(12) Patent Application Publication
(10) Pub. No.: US 2025/0282832 A1
(43) Pub. Date: Sep. 11, 2025

(54) CHIMERIC NUCLEOTIDE SEQUENCE, VECTOR FOR EXPRESSION IN MAMMALS, RNA VACCINE, CHIMERIC FUSION PROTEIN, USE IN THE PRODUCTION OF A VACCINE AGAINST CORONAVIRUS

(71) Applicant: IMUNOTERA SOLUÇÕES TERAPÊUTICAS LTDA., São Paulo (BR)

(72) Inventors: Rúbens Prince Dos Santos ALVES, São Paulo (BR); Luana Raposo De Melo Moraes APS, Cajamar (BR); Bruna Felicio Milazzotto Maldonado Porchia RIBEIRO, São Paulo (BR); Lennon Ramos PEREIRA, São Paulo (BR); Luís Carlos De Souza FERREIRA, Osaco (BR)

(73) Assignee: IMUNOTERA SOLUÇÕES TERAPÊUTICAS LTDA., São Paulo, SP (BR)

(21) Appl. No.: 18/553,324

(22) PCT Filed: Mar. 30, 2022
(86) PCT No.: PCT/BR2022/050112

§ 371 Date: Sep. 29, 2023

(30) Foreign Application Priority Data
Apr. 1, 2021 (BR) 10 2021 006 401-3

(51) Int. Cl. C07K 14/005; A61K 39/00; A61P 31/14; C12N 15/85

(52) U.S. Cl. CPC C07K 14/005; A61P 31/14; C12N 15/85; A61K 2039/53; C12N 2710/16622; C12N 2770/20022; C12N 2770/20034

(57) ABSTRACT

A chimeric nucleotide sequence that corresponds to an encoded fusion protein comprising a polyepitope resulting from selecting and juxtaposing multiple epitopes from a coronavirus protein to induce an immune response in mammals. In one embodiment, said fusion protein comprises: a) a first peptide consisting of epitopes found in the amino acid sequence of replicase polyprotein 1ab (PR1ab); b) a first spacer; c) a modified form of herpes simplex virus type 1 (HSV-1) glycoprotein D (gD). In one embodiment, the replicase polyprotein is defined by SEQ ID NO: 96 flanked by a gD fragment comprising the amino acid sequence defined by SEQ ID NO: 98 in the N-terminal portion and another gD fragment comprising the amino acid sequence defined by SEQ ID NO: 100 in the C-terminal region. Use of the fusion protein has surprising results in inducing cellular and humoral immune responses against coronavirus, SARS-COV-2, and related viruses.

### SEQUENCE LISTING

The instant application contains a Sequence Listing which has been submitted electronically in ASCII format and is hereby incorporated by reference in its entirety. Said ASCII copy, created on Apr. 24, 2024, is named 61094_001U1_SL.txt and is 107,948 bytes in size.

### FIELD OF THE INVENTION

The present invention belongs to the technical field of Biotechnology. More specifically, a chimeric nucleotide sequence is described, the corresponding encoded fusion protein, which comprises a polyepitope resulting from the selection and juxtaposition of multiple epitopes of a coronavirus protein to induce immune response in mammals. In one embodiment, said fusion protein comprises: a) a first peptide consisting of epitopes present in the amino acid sequence of replicase polyprotein 1ab (PR1ab); b) a first spacer; c) a modified form of the glycoprotein D (gD) of herpes simplex type 1 (HSV-1). In one embodiment, the replicase polyprotein is defined by SEQ ID NO: 96 flanked from a gD fragment comprising the amino acid sequence defined by SEQ ID NO: 98 in the N-terminal portion and another gD fragment comprising the amino acid sequence defined by SEQ ID NO: 100 in the C-terminal region. The use of the fusion protein shows surprising results for the induction of cellular and humoral immune response against coronavirus, SARS-COV-2, and related viruses.

### BACKGROUND OF THE INVENTION

The re-emergence of SARS-COV-2 betacoronavirus has so far resulted in more than 107 million infections and more than two million deaths worldwide (Amanat & Krammer, 2020). The rapid spread of this virus associated with a high morbidity rate, as well as the lack of scientifically confirmed therapeutic and/or prophylactic strategies reinforce the urgency of developing vaccines. The pandemic nature of COVID-19 has driven the race to develop therapeutic treatments, so that dozens of drugs and vaccine candidates are being tested in the clinical and preclinical context (W.-H. Chen et al., 2020; Sun et al., 2020).

Symptoms of COVID-19 can range from absent and mild to severe, and includes fever, dry cough, dyspnea, myalgia, tiredness, leukopenia, and even pneumonia. The patient immune system widely contributes to this condition. In more severe cases, a phenomenon called cytokine storm, an abrupt and intense release of pro-inflammatory cytokines (IFN-α, IFN-γ, IL-1β, IL-6, IL-12, IL-18, IL-33, TNF-α, TGF-β) and chemokines (CCL2, CCL3, CCL5, CXCL8, CXCL9, CXCL10) result in an accumulation of fluid in the lungs, multiple failure and death (Li et al., 2020). The virus causing the disease belongs to the same family as MERS-COV and SARS-COV, being responsible for acute respiratory syndromes that have become epidemics in the last two decades.

SARS-COV-2 belongs to the Coronaviridae family, order Nidovirales, which is divided into four genera (Alpha-, Beta-, Gamma-, Delta-Coronavirus), most of which are zoonotic viruses (Grifoni et al., 2020). These viruses are enveloped with positive sense single-stranded RNA, and genomes between 26 and 32 kb with 29.8 kb, the SARS-COV-2 genome has 14 ORFs (Open Reading Frame(s)) that encode 27 proteins. Region 5′ contains the overlapping ORFs orf1ab and orf1a that encode two polyproteins that are processed into 15 non-structural proteins (nsp1-nsp10 and nsp12-nsp16), and region 3′ contains the 4 structural proteins (spike, envelope, membrane, and nucleocapsid), and 8 accessory proteins (3a, 3b, p6, 7a, 7b, 8b, 9b, and orf14) (Wu et al., 2020), as shown in FIG. 1.

Electron microscopy tests showed that SARS-COV and SARS-COV-2 show great similarity and enter the cells using the same receptor, the angiotensin converting enzyme (ACE2), to which the spike glycosylated protein binds and undergoes irreversible conformational changes before entering the cell (Wrapp et al., 2020). S glycoprotein of SARS-COV-2 has 77% similarity to SARS-COV spike and it is a natural target due to its critical role in receptor binding and consequent fusion of the virus membranes and the host, being therefore considered the main antigen (Yuan et al., 2020).

Most vaccine strategies under development target the viral spike protein, which is present on the virion surface and is responsible for the virus interaction with the ACE2 cellular receptor, being, therefore, the main target of neutralizing antibodies (Roper & Rehm, 2009). However, in the natural response developed against coronavirus, there is not always an induction of antigen-specific long-lasting antibodies. Preclinical studies with vaccine strategies based on inactivated SARS-COV-1 showed that, even with the induction of antibodies with neutralizing and protective capacity, some of the immunized animals showed clinical complications after the challenge, with pulmonary injury with eosinophilic infiltration being reported in mice, and increased severity of the disease being reported in ferrets (Tseng et al., 2012; Weingartl et al., 2004). Epidemiological data from the SARS-COV-1 outbreak in China in 2005 suggests that cross-reaction neutralizing antibodies, detectable during the first two weeks of the disease, were correlated with high rates of early mortality by SARS (Yang et al., 2005). Further studies have shown that antibodies generated against S protein variants tend to increase infection of human macrophages by a mechanism known as Antibody-Dependent Enhancement (ADE) (Wang et al., 2014). This phenomenon is the pathogenic basis of the feline infectious peritonitis virus (FIPV, a type II coronavirus) and severe dengue (Tirado & Yoon, 2003).

Based on data from the dengue literature, it is known that when the vaccine response is accompanied by the activation of CD8⁺ T cells, the pathogenic effect of the antibodies is neutralized (Zellweger et al., 2014). In addition, according to previous studies using SARS-COV-1, the adoptive transfer of CD8⁺ T cells promotes viral load reduction, clinical recovery, and protection under experimental conditions (Zhao et al., 2010). In convalescent patients, it was possible to detect CD8⁺ and CD4⁺ memory T cells up to four years after primary infection, which were responsive to in vitro restimulation, and resulted in the high production of cytokines and molecules having an antiviral role, such as IFNγ, perforin and granzyme B (H. Chen et al., 2005). These results show that T cells play a critical role in the mediation of protection against SARS-COV-2.

Cytotoxic T lymphocytes (CTL) recognize antigens as short peptides (usually about 8 to about 10 amino acids in length) presented by class I molecules of the main histocompatibility complex (MHC) (Townsend & Bodmer, 1989). The characterization of these antigen fragments, called immunogenic epitope determinants, has allowed the design of immunization strategies based on the use of vaccines containing such segments (Berzofsky et al., 2001; Melief & Van Der Burg, 2008). As a product of recombinant DNA technology, it was possible to develop a class of new biomolecules with chimeric characteristics and multi-antigen property. By genetically fusing two or more antigenic segments of one or more target proteins, the product obtained may have the immunogenic determinants of the different original target proteins. Thus, the fusion of different epitopes from various sources can induce specific multivalent immunological response against different pathogens/antigens through a unique construction, such strategy being called polyepitope or multiepitope vaccine. In general, vaccine strategies that employ this method have been studied for the treatment of infectious diseases and cancer. There are some works that use this strategy to create COVID-19 vaccines. However, the strategies developed have structural proteins as a target (Kar et al., 2020; Naz et al., 2020; Oladipo et al., 2021; Safavi et al., 2020; Saha et al., 2021; Sohail et al., 2021; Tahir Ul Qamar et al., 2020).

Glycoprotein D (gD) has the ability to interact with the HVEM receptor (Herpesvirus Entry Mediator), which promotes the activation of immune system cells directly by the production of NF-kB (Sciortino et al., 2008), or indirectly by blocking BTLA (B and T Lymphocyte Attenuator) and CD160 binding to this receptor that would result in co-inhibitory signals (Steinberg et al., 2011). It is known in the art the use of antigens fusion to gD for the development of therapeutic vaccines against tumors induced by the human papillomavirus (HPV) both in the form of DNA vaccine (Diniz et al., 2010; Diniz et al., 2013; APS et al., 2015; Diniz et al., 2016) and in the form of purified protein vaccine (Porchia et al., 2011; Porchia et al., 2017), whose results show a significant increase in the induction of local and systemic CD8⁺ T lymphocytes specific against tumor antigens genetically fused to gD.

When searching the state of the art in scientific and patent literatures, the following documents were found that relate to the subject:

PI 0904880-4 discloses the construction of two plasmids, one containing gene expressing oncoprotein E7 of the human papillomavirus type-16 (HPV-16) fused to the glycoprotein D (gD) of herpes simplex type 1 (HSV-1) and another plasmid containing the gene expressing interleukin-2, intended to control tumors.

PI 1003749-7 discloses a hybrid protein, formed by the genetic fusion of oncoprotein E7 of the human papillomavirus type-16 (HPV-16) with a modified form of the glycoprotein D of herpes simplex type 1, intended to be a vaccine adjuvant to other antigens and/or as an active ingredient in pharmaceutical compositions to control tumors.

U.S. Pat. Nos. 8,962,816 and 9,724,406 disclose chimeric proteins, in which one or more antigens are introduced in the C-terminal region of a glycoprotein D (gD) of herpes simplex type 1, intended to more strongly enhance an individual's immune response to the antigen when compared to the protein without fusion with gD. These documents disclose possible applications for vaccines against influenza, malaria, cervical cancer, and HIV/AIDS.

The scientific literature that circumvents the invention, without however anticipating or suggesting it, includes the documents below.
Amanat, F., & Krammer, F. (2020). Perspective SARS-COV-2 Vaccines: Status Report. https://doi.org/10.1016/j.immuni.2020.03.007.
Aps, L. R., Diniz, M. O., Porchia, B. F., Sales, N. S., Moreno, A. C. R., & Ferreira, L. C. (2015). Bacillus subtilis spores as adjuvants for DNA vaccines. Vaccine, 33 (20), 2328-2334. https://doi.org/10.1016/j.vaccine.2015.03.043.
Berzofsky, J. A., Ahlers, J. D., & Belyakov, I. M. (2001). Strategies for designing and optimizing new generation vaccines. In Nature Reviews Immunology (Vol. 1, Issue 3, pp. 209-219). European Association for Cardio-Thoracic Surgery. https://doi.org/10.1038/35105075.
Chen, H., Hou, J., Jiang, X., Ma, S., Meng, M., Wang, B., Zhang, M., Zhang, M., Tang, X., Zhang, F., Wan, T., Li, N., Yu, Y., Hu, H., Yang, R., He, W., Wang, X., & Cao, X. (2005). Response of Memory CD8⁺ T Cells to Severe Acute Respiratory Syndrome (SARS) Coronavirus in Recovered SARS Patients and Healthy Individuals. The Journal of Immunology, 175 (1), 591-598. https://doi.org/10.4049/jimmunol.175.1.591.
Chen, W.-H., Strych, U., Hotez, P. J., & Bottazzi, M. E. (2020). The SARS-COV-2 Vaccine Pipeline: an Overview. Current Tropical Medicine Reports. https://doi.org/10.1007/s40475-020-00201-6.
Diniz, M. O., Lasaro, M. O., Ertl, H. C., & Ferreira, L. C. (2010). Immune responses and therapeutic antitumor effects of an experimental DNA vaccine encoding human papillomavirus type 16 oncoproteins genetically fused to herpesvirus glycoprotein D. Clinical and Vaccine Immunology, 17 (10), 1576-1583. https://doi.org/10.1128/CVI.00264-10.
Diniz, M. O., Cariri, F. A., Aps, L. R., & Ferreira, L. C. (2013). Enhanced therapeutic effects conferred by an experimental DNA vaccine targeting human papillomavirus-induced tumors. Human gene therapy, 24 (10), 861-870. https://doi.org/10.1089/hum.2013.102.
Diniz, M. O., Sales, N. S., Silva, J. R., & Ferreira, L. C. S. (2016). Protection against HPV-16-Associated Tumors Requires the Activation of CD8+ Effector Memory T Cells and the Control of Myeloid-Derived Suppressor Cells. Molecular cancer therapeutics, 15 (8), 1920-1930. https://doi.org/10.1158/1535-7163.
Dos Santos Franco, L., Oliveira Vidal, P., & Amorim, J. H. (2017). In silico design of a Zika virus non-structural protein 5 aiming vaccine protection against zika and dengue in different human populations. Journal of Biomedical Science, 24 (1), 1-10. https://doi.org/10.1186/s12929-017-0395-z.
Grifoni, A., Weiskopf, D., Ramirez, S. I., Mateus, J., Dan, J. M., Moderbacher, C. R., Rawlings, S. A., Sutherland, A., Premkumar, L., Jadi, R. S., Marrama, D., de Silva, A. M., Frazier, A., Carlin, A. F., Greenbaum, J. A., Peters, B., Krammer, F., Smith, D. M., Crotty, S., & Sette, A. (2020). Targets of T Cell Responses to SARS-COV-2 Coronavirus in Humans with COVID-19 Disease and Unexposed Individuals. Cell, 181 (7), 1489-1501.e15. https://doi.org/10.1016/j.cell.2020.05.015.
Kar, T., Narsaria, U., Basak, S., Deb, D., Castiglione, F., Mueller, D. M., & Srivastava, A. P. (2020). A candidate multi-epitope vaccine against SARS-CoV-2. Scientific Reports, 10 (1). https://doi.org/10.1038/s41598-020-67749-1.
Li, X., Geng, M., Peng, Y., Meng, L., & Lu, S. (2020). Molecular immune pathogenesis and diagnosis of COVID-19. In Journal of Pharmaceutical Analysis (Vol. 10, Issue 2, pp. 102-108). Xi'an Jiaotong University. https://doi.org/10.1016/j.jpha.2020.03.001.
Melief, C. J. M., & Van Der Burg, S. H. (2008). Immunotherapy of established (pre) malignant disease by synthetic long peptide vaccines. In Nature Reviews Cancer (Vol. 8, Issue 5, pp. 351-360). Nat Rev Cancer. https://doi.org/10.1038/nrc2373.
Naz, A., Shahid, F., Butt, T. T., Awan, F. M., Ali, A., & Malik, A. (2020). Designing Multi-Epitope Vaccines to Combat Emerging Coronavirus Disease 2019 (COVID-19) by Employing Immuno-Informatics Approach. Frontiers in Immunology, 11, 1663. https://doi.org/10.3389/fimmu.2020.01663.
Porchia, B. F., Diniz, M. O., Cariri, F. A., Santana, V. C., Amorim, J. H., Balan, A., & Ferreira, L. C. S. (2011). Purified herpes simplex type 1 glycoprotein D (gD) genetically fused with the type 16 human papillomavirus E7 oncoprotein enhances antigen-specific CD8+ T cell responses and confers protective antitumor immunity. Molecular pharmaceutics, 8 (6), 2320-2330. https://doi.org/10.1021/mp200194s.
Porchia, B. F., Moreno, A. C. R., Ramos, R. N., Diniz, M. O., de Andrade, L. H. T., Rosa, D. S., . . . & Ferreira, L. C. S. (2017). Herpes simplex virus glycoprotein D targets a specific dendritic cell subset and improves the performance of vaccines to human papillomavirus-associated tumors. Molecular cancer therapeutics, 16 (9), 1922-1933. https://doi.org/10.1158/1535-7163.
Oladipo, E. K., Ajayi, A. F., Onile, O. S., Ariyo, O. E., Jimah, E. M., Ezediuno, L. O., Adebayo, O. I., Adebayo, E. T., Odeyemi, A. N., Oyeleke, M. O., Oyewole, M. P., Oguntomi, A. S., Akindiya, O. E., Aremu, V. O., Aboderin, D. O., & Oloke, J. K. (2021). Designing a conserved peptide-based subunit vaccine against SARS-COV-2 using immunoinformatics approach. In Silico Pharmacology, 9 (1). https://doi.org/10.1007/s40203-020-00062-x.
Roper, R. L., & Rehm, K. E. (2009). SARS vaccines: where are we? Expert Review of Vaccines, 8 (7), 887-898. https://doi.org/10.1586/erv.09.43.
Safavi, A., Kefayat, A., Mahdevar, E., Abiri, A., & Ghahremani, F. (2020). Exploring the out of sight antigens of SARS-COV-2 to design a candidate multi-epitope vaccine by utilizing immunoinformatics approaches. Vaccine, 38 (48), 7612-7628. https://doi.org/10.1016/j.vaccine.2020.10.016.
Saha, R., Ghosh, P., & Burra, V. L. S. P. (2021). Designing a next generation multi-epitope based peptide vaccine candidate against SARS-COV-2 using computational approaches. 3 Biotech, 11 (2), 47. https://doi.org/10.1007/s13205-020-02574-x.
Sciortino, M. T., Medici, M. A., Marino-Merlo, F., Zaccaria, D., Giuffrè-Cuculletto, M., Venuti, A., Grelli, S., & Mastino, A. (2008). Involvement of HVEM receptor in activation of nuclear factor KB by herpes simplex virus 1 glycoprotein D. Cellular Microbiology, 10 (11), 2297-2311. https://doi.org/10.1111/j.1462-5822.2008.01212.x.
Sohail, M. S., Ahmed, S. F., Quadeer, A. A., & Mckay, M. R. (2021). In silico T cell epitope identification for SARS-COV-2: Progress and perspectives. In Advanced Drug Delivery Reviews (Vol. 171, pp. 29-47). Elsevier B.V. https://doi.org/10.1016/j.addr.2021.01.007.
Steinberg, M. W., Cheung, T. C., & Ware, C. F. (2011). The signaling networks of the herpesvirus entry mediator (TNFRSF14) in immune regulation. Immunological Reviews, 244 (1), 169-187. https://doi.org/10.1111/j.1600-065X.2011.01064.x.
Sun, K., Chen, J., & Viboud, C. (2020). Early epidemiological analysis of the coronavirus disease 2019 outbreak based on crowdsourced data: a population-level observational study. The Lancet Digital Health, 2 (4), e201-e208. https://doi.org/10.1016/S2589-7500 (20) 30026-1.
Tahir Ul Qamar, M., Shahid, F., Aslam, S., Ashfaq, U. A., Aslam, S., Fatima, I., Fareed, M. M., Zohaib, A., & Chen, L. L. (2020). Reverse vaccinology assisted designing of multiepitope-based subunit vaccine against SARS-COV-2. Infectious Diseases of Poverty, 9 (1). https://doi.org/10.1186/s40249-020-00752-w.
Tirado, S. M. C., & Yoon, K. J. (2003). Antibody-dependent enhancement of virus infection and disease. Viral Immunology, 16 (1), 69-86. https://doi.org/10.1089/088282403763635465.
Townsend, A., & Bodmer, H. (1989). Antigen recognition by class I-restricted T lymphocytes. In Annual Review of Immunology (Vol. 7, pp. 601-624). Annu Rev Immunol. https://doi.org/10.1146/annurev.iy.07.040189.003125.
Tseng, C.-T., Sbrana, E., Iwata-Yoshikawa, N., Newman, P. C., Garron, T., Atmar, R. L., Peters, C. J., & Couch, R. B. (2012). Immunization with SARS Coronavirus Vaccines Leads to Pulmonary Immunopathology on Challenge with the SARS Virus. PLOS ONE, 7 (4), e35421. https://doi.org/10.1371/journal.pone.0035421.
Wang, S. F., Tseng, S. P., Yen, C. H., Yang, J. Y., Tsao, C. H., Shen, C. W., Chen, K. H., Liu, F. T., Liu, W. T., Chen, Y. M. A., & Huang, J. C. (2014). Antibody-dependent SARS coronavirus infection is mediated by antibodies against spike proteins. Biochemical and Biophysical Research Communications, 451 (2), 208-214. https://doi.org/10.1016/j.bbrc.2014.07.090.
Weingartl, H., Czub, M., Czub, S., Neufeld, J., Marszal, P., Gren, J., Smith, G., Jones, S., Proulx, R., Deschambault, Y., Grudeski, E., Andonov, A., He, R., Li, Y., Copps, J., Grolla, A., Dick, D., Berry, J., Ganske, S., . . . Cao, J. (2004). Immunization with Modified Vaccinia Virus Ankara-Based Recombinant Vaccine against Severe Acute Respiratory Syndrome Is Associated with Enhanced Hepatitis in Ferrets. Journal of Virology, 78 (22), 12672-12676. https://doi.org/10.1128/JVI.78.22.12672-12676.2004.
Wrapp, D., Wang, N., Corbett, K. S., Goldsmith, J. A., Hsieh, C. L., Abiona, O., Graham, B. S., & Mclellan, J. S. (2020). Cryo-EM structure of the 2019-nCOV spike in the prefusion conformation. In bioRxiv. bioRxiv. https://doi.org/10.1101/2020.02.11.944462.
Wu, F., Zhao, S., Yu, B., Chen, Y. M., Wang, W., Song, Z. G., Hu, Y., Tao, Z. W., Tian, J. H., Pei, Y. Y., Yuan, M. L., Zhang, Y. L., Dai, F. H., Liu, Y., Wang, Q. M., Zheng, J. J., Xu, L., Holmes, E. C., & Zhang, Y. Z. (2020). A new coronavirus associated with human respiratory disease in China. Nature, 579 (7798), 265-269. https://doi.org/10.1038/s41586-020-2008-3.
Yang, Z. Y., Werner, H. C., Kong, W. P., Leung, K., Traggiai, E., Lanzavecchia, A., & Nabel, G. J. (2005). Evasion of antibody neutralization in emerging severe acute respiratory syndrome coronaviruses. Proceedings of the National Academy of Sciences of the United States of America, 102 (3), 797-801. https://doi.org/10.1073/pnas.0409065102.
Yuan, M., Wu, N. C., Zhu, X., Lee, C. C. D., So, R. T. Y., Lv, H., Mok, C. K. P., & Wilson, I. A. (2020). A highly conserved cryptic epitope in the receptor binding domains of SARS-COV-2 and SARS-COV. Science, 368 (6491), 630-633. https://doi.org/10.1126/science.abb7269.
Zellweger, R. M., Eddy, W. E., Tang, W. W., Miller, R., & Shresta, S. (2014). CD8+ T Cells Prevent Antigen-Induced Antibody-Dependent Enhancement of Dengue Disease in Mice. The Journal of Immunology, 193 (8), 4117-4124. https://doi.org/10.4049/jimmunol.1401597.
Zhao, J., Zhao, J., & Perlman, S. (2010). T cell responses are required for protection from clinical disease and for virus clearance in severe acute respiratory syndrome coronavirus-infected mice. Journal of Virology, 84 (18), 9318-9325. https://doi.org/10.1128/JVI.01049-10.

From what is evident from the literature researched, no documents were found anticipating or suggesting the teachings of the present invention, so that the solution proposed herein has, in the eyes of the inventors, industrial applicability, novelty and inventive activity relative to the state of the art. One of the objects of the present invention provides an alternative vaccine and/or an alternative to obtain vaccines against Coronavirus, SARS-COV-2, or COVID-19, and related viruses.

### SUMMARY OF THE INVENTION

The present invention provides a chimeric nucleotide sequence comprising a polyepitope resulting from the selection and juxtaposition of multiple epitopes of a coronavirus protein to induce immune response in mammals.

The DNA or RNA nucleotide sequences encoding the aforementioned polypeptide are also the object of the invention, as well as the mammalian expression vectors comprising said nucleotide sequences.

The present invention provides an alternative vaccine and/or an alternative to obtain cell and/or humoral immunity-inducing vaccines against Coronavirus, SARS-COV-2, or COVID-19, and related viruses. In one embodiment, the antigenic elements are non-structural proteins of the virus.

In one embodiment, the present invention provides a vaccine to induce a combined immunological response of CD8⁺ T cells producing antiviral molecules in addition to the humoral response against SARS-COV-2.

The in vivo use of the present invention provides a surprising potential to promote viral load reduction, clinical recovery and protection against coronavirus, SARS-COV-2, or COVID-19, and related viruses, among other advantages.

In one embodiment, the antigenic elements, or targets of the vaccine of the invention are found in the non-structural proteins encoded by the ORF1ab of the Brazilian strain of SARS-COV-2, in contrast to the technologies existing in the art that focus mainly on the virus Spike protein (S protein).

In one embodiment, the present invention provides the association of immunogenic epitopes of these regions compared with the HLAs (human leukocyte antigen system) most frequently found in the Brazilian population, being: C*02:02, A*32:01, A*68:02, A*01:01, B*08:01, A*65:02, B*51:01, B*35:01; where each allele is frequent in at least 3% of the population of Brazil. In other embodiments, the association of immunogenic epitopes is compared with HLAs of specific ethnic groups and customized to different populations.

In one embodiment, separators are used, preferably defined by the amino acid sequence defined by SEQ ID NO: 94 added between the peptides. Among other reasons, these separators may facilitate the structuring of the antigen after the intracellular translation into the expression and/or to facilitate antigen processing.

In one embodiment, the sequence containing the multiepitope is inserted in the sequence of the glycoprotein D (gD) of the herpesvirus. Among other advantages, gD has an adjuvant effect for the activation of T lymphocytes, related to its ability to interact with HVEM (Herpesvirus Entry Mediator) receptor that promotes activation of the immune system cells.

The present invention presents the following objects:

In a first object, the present invention provides a chimeric nucleotide sequence comprising the sequences encoding, in phase, a plurality of epitopes of a coronavirus protein.

In one embodiment, said coronavirus protein is a non-structural protein of the virus, such as the replicase protein 1ab. In one embodiment, the sequences encoding the replicase 1ab multiepitope are aligned in phase, so that the encoded product is a chimeric protein of a replicase 1ab polyepitope.

In one embodiment, said nucleotide sequence additionally comprises a nucleotide sequence that encodes a modified form of the glycoprotein D (gD) of herpes simplex type 1 (HSV-1).

In one embodiment, said nucleotide sequence additionally comprises a nucleotide sequence encoding a first spacer polypeptide.

In one embodiment, said nucleotide sequence comprises:
a) a nucleotide sequence that encodes a first peptide comprising epitopes found in the amino acid sequence of the replicase polyprotein 1ab (PR1ab);
b) a nucleotide sequence encoding a first spacer polypeptide; and
c) a nucleotide sequence that encodes a modified form of the glycoprotein D (gD) of herpes simplex type 1 (HSV-1).

In one embodiment, the nucleotide sequence encoding the polyepitope comprises a phased combination of the nucleotide sequences selected from SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91.

In one embodiment the nucleotide sequence encoding the polyepitope is defined by SEQ ID NO: 95.

In one embodiment, the nucleotide sequence encoding the modified form of the glycoprotein D (gD) of herpes simplex type 1 (HSV-1) is defined by SEQ ID NO: 97 and/or SEQ ID NO: 99.

In a second object, an expression vector is provided, which comprises:
the aforementioned chimeric nucleotide sequence encoding the polyepitope; and
one or more expression promoters functionally linked to said nucleotide sequence.

In one embodiment, the polypeptide sequence of the polyepitope comprises a combination of the polypeptide sequences selected from SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92.

In a third object, an RNA vaccine is provided, which comprises an RNA sequence that encodes the aforementioned polyepitope.

In one embodiment, said RNA sequence comprises a combination of the RNA sequences selected from SEQ ID NO: 113 to SEQ ID NO: 158. In one embodiment, said RNA sequence additionally comprises SEQ ID NO: 105. In one embodiment, said RNA sequence additionally comprises SEQ ID NO: 161 and/or SEQ ID NO: 162.

In a fourth object, the present invention provides a chimeric polypeptide sequence comprising a plurality of epitopes of a coronavirus protein.

In one embodiment, said coronavirus protein is a non-structural protein of the virus, such as the replicase protein 1ab. In one embodiment, multiple epitopes of replicase 1ab are aligned, thus forming a chimeric protein of a replicase 1ab polyepitope.

In one embodiment, said polypeptide sequence additionally comprises a polypeptide sequence of a modified form of the glycoprotein D (gD) of herpes simplex type 1 (HSV-1).

In one embodiment, said polypeptide sequence additionally comprises a sequence of a first spacer polypeptide.

In one embodiment, the chimeric fusion protein comprises:
a) a first peptide consisting of epitopes found in the amino acid sequence of the replicase polyprotein 1ab (PR1ab);
b) a first spacer; and
c) modified form of glycoprotein D (gD) of herpes simplex type 1 (HSV-1).

In one embodiment, the peptide sequence comprising the polyepitope is defined by SEQ ID NO: 96.

In one embodiment, the peptide sequence of the modified form of gD is defined by SEQ ID NO: 98 and/or SEQ ID NO: 100.

As a fifth object, it is provided the use of the chimeric nucleotide sequence, the RNA chimeric sequence or the fusion protein for the preparation of a vaccine for coronavirus, SARS-COV-2, and/or related viruses.

These and other objects of the invention will be immediately valued by those skilled in the art and will be described in detail below.

### BRIEF DESCRIPTION OF THE FIGURES

The following figures are shown:

FIG. 1 depicts the organization of the SARS-COV-2 genome showing the arrangement of the ORFS (Open Reading Frame) and the distribution of structural and non-structural proteins (Wu et al., 2020).

FIG. 2 shows a) a scheme of the gene structure of SARS-COV-2 and b) the target alleles of two regions: NSa and NSb.

FIG. 3 shows the analysis of the in vitro expression of vaccine antigens. After 36 hours of transfection with each vaccine plasmid, the cells were subjected to gD protein immunodetection. In (a), a flow cytometry analysis was performed. In (b), after fixing the cell monolayer on the plate, immunofluorescence was performed with glycoprotein D antibody, where cells positive for the expression of the recombinant antigen encoded by DNA vaccines are marked in green and the nucleus of the cells are marked in blue, with an increase of 20× or 40×.

FIG. 4 shows the evaluation of cellular response induced by DNA vaccines associated with ICS electroporation. 5-6 week-old C57BL/6 mice received two intramuscular doses of the vaccines (50 ug pgDPOLYEP, 50 ug NSA+50 ug NSB or 50 ug pcDNA3.1) together with electroporation, with two weeks between the applications. Two weeks after the last dose, the animals were euthanized and the spleen cells were used for ex vivo stimulation with SARS-CoV-2 peptides for functional evaluation (a). The subpopulation of CD8 T lymphocytes (CD3⁺CD8⁺) that experienced the encounter with the in vivo antigen (CD49d⁺CD11a^{hi}) was evaluated for the relative frequency of IFNγ (a)-, TNF (b)-, and IFNγ+TNF (c)-producing cells, as well as the absolute count of the IFNγ (d)-, TNF (e)-, and IFNγ+TNF (f)-producing cells. Data represents the mean±SEM and one-tailed Mann-Whitney U test, where *p<0.05 **p<0.01, ***p<0.001, ****p<0.0001.

FIG. 5 shows the evaluation of cellular response induced by DNA vaccines associated with ELISPOT electroporation. 5-6 week-old C57BL/6 mice received two intramuscular doses of the vaccines (50 ug pgDPOLYEP, 50 ug NSA+50 ug NSB or 50 ug pcDNA3.1) together with electroporation, with two weeks between the applications. Two weeks after the last dose, the animals were euthanized and the spleen cells were used for ex vivo stimulation with SARS-CoV-2 peptides for evaluating IFN-γ production. Data represents the mean±SEM and the one-tailed Mann-Whitney U test, where *p<0.05 **p<0.01, ****p<0.0001.

FIG. 6 shows a schematic representation of an embodiment of the invention, in which: in A) an embodiment of the chimeric polypeptide sequence of the invention (POLYEP) is schematically shown, which comprises the polypeptide sequences of a polyepitope of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92; in B) the chimeric nucleotide sequence of the invention is shown in the embodiment which comprises SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91 and two nucleotide sequences of gD fragments, one in 5′ and the other in 3′, these sequences being functionally linked to a plasmid expression vector (pgDPOLYEP) functioning as a DNA vaccine and comprising a CMV promoter; in C) an embodiment of the chimeric polypeptide sequence of the invention (gDPOLYEP) is schematically shown, which comprises the polypeptide sequences of a polyepitope of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92 flanked from a gD fragment comprising the amino acid sequence defined by SEQ ID NO: 98 in the N-terminal portion and another gD fragment comprising the amino acid sequence defined by SEQ ID NO: 100 in the C-terminal region.

### DETAILED DESCRIPTION OF THE INVENTION

The present invention provides an alternative vaccine and/or an alternative to obtain cell and/or humoral immunity-inducing vaccines against Coronavirus, SARS-COV-2, or COVID-19, and related viruses.

The present invention provides a chimeric nucleotide sequence comprising the sequences encoding, in phase, a plurality of epitopes of a coronavirus protein, and/or of the fusion chimeric polypeptide.

In one embodiment, said coronavirus protein is a non-structural protein of the virus, such as the replicase protein 1ab. In one embodiment, the sequences encoding multiple epitopes of replicase 1ab are aligned in phase. The encoded product is a chimeric protein from a replicase 1ab polyepitope.

In one embodiment, the nucleotide sequence encoding the polyepitope comprises a phased combination of the nucleotide sequences selected from SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91.

In one embodiment, the nucleotide sequence encoding the polyepitope is defined by SEQ ID NO: 95.

In one embodiment, said nucleotide sequence additionally comprises a nucleotide sequence that encodes a modified form of the glycoprotein D (gD) of herpes simplex type 1 (HSV-1).

In one embodiment, the nucleotide sequence encoding the modified form of the glycoprotein D (gD) of herpes simplex type 1 (HSV-1) is defined by SEQ ID NO: 97 and/or SEQ ID NO: 99.

In one embodiment, said nucleotide sequence additionally comprises a nucleotide sequence encoding a first spacer polypeptide.

In one embodiment, said chimeric nucleotide sequence comprises:
a) a nucleotide sequence that encodes a first peptide comprising epitopes found in the amino acid sequence of the replicase polyprotein 1ab (PR1ab);
b) a nucleotide sequence encoding a first spacer polypeptide; and
c) a nucleotide sequence that encodes a modified form of the glycoprotein D (gD) of herpes simplex type 1 (HSV-1).

In one embodiment, said chimeric nucleotide sequence is defined by SEQ ID NO: 101.

A mammalian expression vector, comprising said chimeric nucleotide sequence, is also part of the objects of the invention.

In one embodiment, said expression vector is a DNA vaccine to induce cellular and humoral immunity, having as antigenic targets the non-structural proteins of the virus, inducing a combined immunological response of CD8⁺ T cells producing antiviral molecules in addition to the humoral response against SARS-COV-2.

In one embodiment, the use of the invention surprisingly promotes viral load reduction, clinical recovery and protection against coronavirus, SARS-COV-2, or COVID-19, and related viruses.

In one embodiment, said SARS-COV-2 antigens were genetically fused to another viral protein, the glycoprotein D (gD) of the Herpes Simplex Virus Type 1 (HSV-1). This fusion showed an increase in antigens immunogenicity. gD showed adjuvant effect for the activation of T lymphocytes.

The present invention provides an RNA vaccine comprising an RNA sequence that encodes a plurality of epitopes of a coronavirus non-structural protein.

In one embodiment, said vaccine comprises the sequences encoding multiple epitopes of replicase 1ab aligned in phase.

In one embodiment, said vaccine comprises a combination of the RNA sequences selected from SEQ ID NO: 113 to SEQ ID NO: 158.

The present invention provides a chimeric fusion protein comprising a plurality of epitopes of a coronavirus non-structural protein.

The present invention provides a chimeric fusion protein comprising a combination of the peptide sequences of replicase 1ab selected from SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92.

In one embodiment, said chimeric fusion protein comprises SEQ ID NO: 96.

In one embodiment, said chimeric fusion protein additionally comprises a polypeptide sequence of a modified form of the glycoprotein D (gD) of herpes simplex type 1 (HSV-1).

In one embodiment, said polypeptide sequence of the modified form of the glycoprotein D (gD) of herpes simplex type 1 (HSV-1) is defined by SEQ ID NO: 98 and/or SEQ ID NO: 100.

In one embodiment, said chimeric fusion protein additionally comprises a sequence of a first spacer polypeptide, preferably defined by SEQ ID NO: 94.

In one embodiment, said fusion protein comprises:
a) a first peptide consisting of epitopes found in the amino acid sequence of the replicase polyprotein 1ab (PR1ab);
b) a first spacer;
c) modified form of glycoprotein D (gD) of herpes simplex type 1 (HSV-1).

In one embodiment, the first peptide consists in epitopes present in the amino acid sequence of the NSa regions as defined in SEQ ID NO: 110 and/or NSb as defined in SEQ ID NO: 112 in PR1ab.

In one embodiment, the spacer consists in the amino acid sequence GGGS as defined in SEQ ID NO: 94.

In one embodiment, the invention provides a vector comprising the sequences encoding the fusion protein defined above. In one embodiment, said vector additionally comprises a promoter region and Kozak sequence, in which the promoter is preferably CMV and said vector is pcDNA3.1.

The results shown in this patent application demonstrate that, surprisingly, mice that received the vaccine of the invention, in the embodiment of pgDPOLYEP DNA, exhibited CD8⁺ T cells with a dominant Th1 phenotype once stimulated with peptides (pep), with significantly higher numbers of IFNγ-, TNF- and IL-2-producing cells, compared to the other DNA vaccines containing the NSA and NSB portion or the pcDNA3.1 vector.

The vaccine of the invention, in the embodiment comprising the chimeric nucleotide sequences encoding the fusion protein (called pgDPOLYEP), induced the highest production of IFNγ when compared to the formulations where pgDNSA+pgDNSB or the empty vaccine vector pDNA3.1 was administered. Altogether, the results show that pgDPOLYEP is immunogenic capable of inducing cell-type immune response by inducing IFNγ- and TNF-producing CD8⁺ T lymphocytes.

### EXAMPLES

The examples shown here are intended only to exemplify one of the numerous ways to realize the invention, but without limiting the scope of the invention.

### Example 1—Construction of the pgDPOLYEP Vaccine

For the choice of target sequences within the non-structural proteins of Covid19, an associative analysis was performed between the predicted epitopes and the most frequent HLA type I molecules found in the Brazilian population. For the same chosen regions, the linking epitopes of MHC H-2Kb (C57BL/6) were predicted. The allele frequency of HLA class I found in the Brazilian population recovered was from the NCBI database (https://www.ncbi.nlm.nih.gov/projects/gv/mhc/ihwg.cgi). Frequent alleles in at least 3% of the Brazilian population were selected (HLA: C*02:02, A*32:01, A*68:02, A*01:01, B*08:01, A*65:02, B*51:01, B*35:01) (Dos Santos Franco et al., 2017). The epitopes within the amino acid sequence of the replicase 1ab (PR1ab) polyprotein were predicted using the IEDB (Immune epitope database) analysis resource (http://tools.immuneepitope.org/mhci/), according to the recommended method. The epitopes predicted within the PR1ab were classified by their percentage value and for each HLA molecule considered, and the best 5 ones were selected with a percentile less than 0.5. Epitopes with 8, 9, 10, and 11 amino acids in length for the HLA class I were considered.

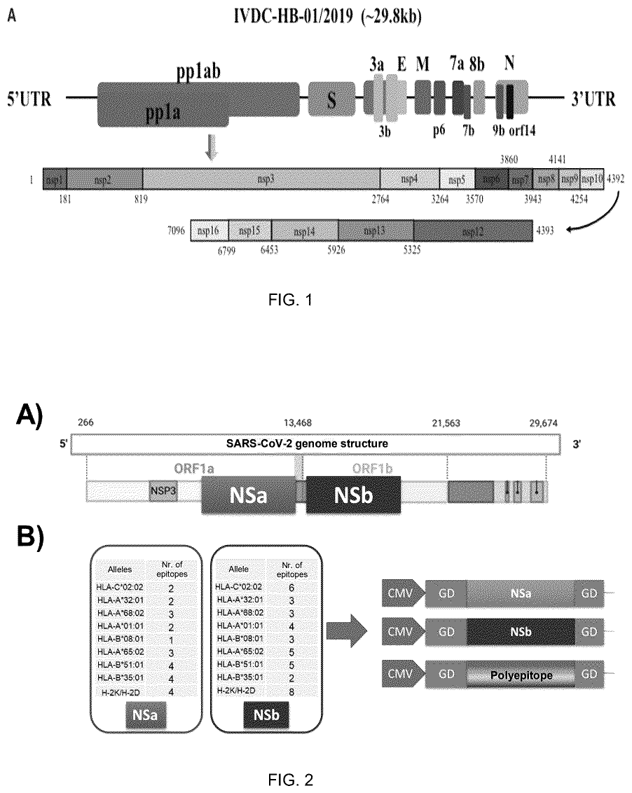

After the associative analysis, two regions in PR1ab were selected that concentrate most of the target sequences for humans and mice (NSa and NSb). The nucleotide sequence encoding both regions, as well as that of the peptides predicted in sequence and linked by the sequence encoding the GGGS amino acids (SEQ ID NO: 94), were inserted in the vaccine vector pcDNA3.1 in fusion with the glycoprotein D (gD) of the herpesvirus, with the addition of Kozak sequence and under the CMV promoter control. The three plasmids (pCovid_NSa, pCovid_NSb, and pCovid_Polyept), respectively comprising Seq ID 1, 2, and 3, as well as the corresponding encoded peptides (purified by reverse phase HPLC with >95% purity), were synthesized and commercially obtained (Genscript), FIG. 2, and Table 1.

TABLE 1
Peptides selected in PR1ab of SARS-CoV-2 (GeneBank: MT126808.1)

| SEQ | | | | | MHC/HLA | |
|---|---|---|---|---|---|---|
| ID | | | Size | Corresponding | biding | |
| No | Start | End | (mer) | protein | prediction | |
| 2 | 2556 | 2564 | 9 | NSP3 | H-2-Db | pNSA |
| 4 | 2586 | 2593 | 8 | NSP3 | HLA-B*35:01 | |
| 6 | 2594 | 2604 | 11 | NSP3 | HLA-C*02:02 | |
| 8 | 2782 | 2796 | 15 | NSP4 | HLA-A*68:02 + | |
| | | | | | HLA-C*02:02 | |
| 10 | 2889 | 2897 | 9 | NSP4 | HLA-A*01:01 | |
| 12 | 2930 | 2938 | 9 | NSP4 | HLA-B*51:01 | |
| 14 | 3064 | 3072 | 9 | NSP4 | HLA-B*08:01 | |
| 16 | 3127 | 3144 | 18 | NSP4 | H-2-Db + HLA- | |
| | | | | | A*32:01 + 01 | |
| | | | | | HLA-B*35:01 | |
| 18 | 3164 | 3172 | 9 | NSP4 | HLA-A*32:01 | |
| 20 | 3179 | 3187 | 9 | NSP4 | HLA-A*65:02 | |
| 22 | 3190 | 3198 | 9 | NSP4 | HLA-B*08:01 | |
| 24 | 3202 | 3210 | 9 | NSP4 | H-2-Db | |
| 26 | 3361 | 3369 | 9 | NSP5/3C-like | HLA-B*08:01 | |
| | | | | proteinase | | |
| 28 | 3420 | 3428 | 9 | NSP5/3C-like | H-2-Db | |
| | | | | proteinase | | |
| 30 | 3437 | 3445 | 9 | NSP5/3C-like | HLA-A*01:01 | |
| | | | | proteinase | | |
| 32 | 3468 | 3476 | 9 | NSP5/3C-like | HLA-B*51:01 | |
| | | | | proteinase | | |
| 34 | 3612 | 3634 | 23 | NSP6 | HLA-B*35:01 + | |
| | | | | | HLA-A*68:02 + | |
| | | | | | HLA-B*08:01 | |
| 36 | 3641 | 3663 | 23 | NSP6 | 01 HLA-B*35:01 + | |
| | | | | | H-2-Db + HLA- | |
| | | | | | B*51:01 | |
| 38 | 4625 | 4633 | 9 | RpRd/NSP12 | H-2-Db | pNSB |
| 40 | 4724 | 4734 | 11 | RpRd/NSP12 | HLA-A*32:01 + | |
| | | | | | HLA-C*02:02 | |
| 42 | 4764 | 4772 | 9 | RpRd/NSP12 | HLA-B*35:01 | |
| 44 | 4789 | 4799 | 11 | RpRd/NSP12 | HLA-A*68:02 + | |
| | | | | | H-2-Db | |
| 46 | 4842 | 4850 | 9 | RpRd/NSP12 | HLA-A*01:01 | |
| 48 | 4863 | 4871 | 9 | RpRd/NSP12 | HLA-C*02:02 | |
| 50 | 4905 | 4913 | 9 | RpRd/NSP12 | HLA-A*65:02 | |
| 52 | 4920 | 4934 | 15 | RpRd/NSP12 | HLA-A*65:02 + | |
| | | | | | HLA-C*02:02 | |
| 54 | 4961 | 4969 | 9 | RpRd/NSP12 | HLA-A*65:02 | |
| 56 | 5016 | 5024 | 9 | RpRd/NSP12 | H-2-Db | |
| 58 | 5039 | 5047 | 9 | RpRd/NSP12 | HLA-B*08:01 | |
| 60 | 5130 | 5148 | 19 | RpRd/NSP12 | HLA-A*01:01 + | |
| | | | | | H-2-Db + HLA- | |
| | | | | | C*02:02 + HLA- | |
| | | | | | B*08:01 | |
| 62 | 5221 | 5229 | 9 | RpRd/NSP12 | HLA-B*51:01 | |
| 64 | 5246 | 5254 | 9 | RpRd/NSP12 | HLA-B*08:01 + | |
| | | | | | H-2-Db | |
| 66 | 5274 | 5282 | 9 | RpRd/NSP12 | HLA-A*65:02 | |
| 68 | 5299 | 5307 | 9 | RpRd/NSP12 | HLA-A*01:01 | |
| 70 | 5381 | 5389 | 9 | Helicase/NSP13 | HLA-A*01:01 | |
| 72 | 5392 | 5400 | 9 | Helicase/NSP13 | HLA-A*65:02 | |
| 74 | 5405 | 5413 | 9 | Helicase/NSP13 | HLA-B*51:01 | |
| 76 | 5455 | 5463 | 9 | Helicase/NSP13 | HLA-A*65:02 | |
| 78 | 5467 | 5475 | 9 | Helicase/NSP13 | HLA-A*68:02 | |
| 80 | 5516 | 5524 | 9 | Helicase/NSP13 | HLA-A*32:01 | |
| 82 | 5533 | 5541 | 9 | Helicase/NSP13 | HLA-C*02:02 | |
| 84 | 5557 | 5565 | 9 | Helicase/NSP13 | HLA-B*51:01 | |
| 86 | 5596 | 5604 | 9 | Helicase/NSP13 | H-2-Db | |
| 88 | 5615 | 5623 | 9 | Helicase/NSP13 | HLA-B*35:01 | |
| 90 | 5630 | 5638 | 9 | Helicase/NSP13 | HLA-A*68:02 | |
| 92 | 5649 | 5657 | 9 | Helicase/NSP13 | HLA-B*51:01 | |

### Example 2—Evaluation of gDPOLYEP Expression In Vitro

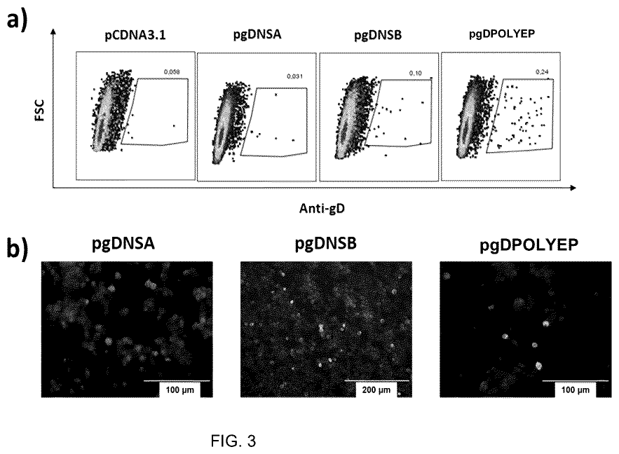

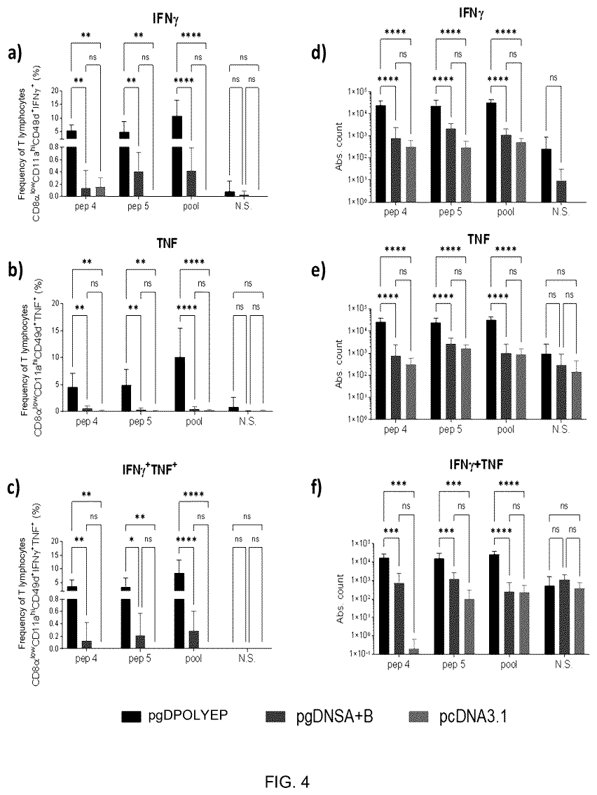

Vaccine plasmids were validated by transfection in eukaryotic cells followed by immunodetection of target proteins. For this, HEK293-T cells were cultured (DMEM+10% SFB) in 24-well plates (10×5 cells/well) for 24 hours (37° C., 5% CO2). After reaching confluence of 70-80%, the cells were transfected with the recombinant plasmids (pgDNSA, pgDNSB, and pgDPOLYEP) or controls (pCDNA3.1) using the Lipofectamine 2000 Kit (Thermo Fisher), according to the manufacturer's instructions. After 24-48 hours of transfection, the cells were trypsinized, washed with PBS solution (twice) and fixed/permeated with Cytofix buffer (BD Biosciences) according to the manufacturer's instructions. After a new washing cycle, the cells were incubated (30 min. on ice) with previously diluted monoclonal anti-gD antibody. After the washing cycle, the cells were incubated with anti-IgG mouse/human antibody conjugated to fluorochrome AlexaFluor 488 (Invitrogen) for 30 min. on ice. After a new washing cycle, the cells were resuspended in PBS+2% SFB and evaluated by flow cytometry in a BD LSRFortessa™ (BD Biosciences) equipment. Data obtained was analyzed using FlowJo v.10 software (TreeStar, OR, USA). As shown in FIG. 3a, from the immunodetection of gD it was possible to determine the in vitro expression of the vaccine antigen gDPOLYEP, as a result of the increase in the frequency of cells marked as positive for gD expression.

The detection of target protein expression in transfected cells was also evaluated by the immunofluorescence technique. For this purpose, transfected cells as described above were directly fixed with 4% paraformaldehyde (PFA) solution in PBS (300 μl/well) for 15 minutes at room temperature (RT). After fixing, the cells were permeated with 0.1% Triton X-100 solution in PBS (300 μl/well) for 10 minutes at RT. Then, the cells were washed twice with PBS and blocked with a 2% BSA solution in PBS (30 min., RT). After the blocking step, anti-gD mAb or hyperimmune serum previously diluted in a blocking solution (200 μl/well) were added to the wells. After 1 hour, the wells were washed (3 times) with PBS, the cells were incubated (45 min., RT) with mouse/human anti-lgG antibody conjugated with AlexaFluor 488 (Invitrogen), under agitation. After a new washing cycle, the cells were incubated (20 min., RT) with Hoechst 33342 nucleus stain (Life Technologies) diluted (1/500) in PBS (200 μL/well). After a new washing cycle, the cells were visualized using an Evos FL (Thermo Fisher Scientific) immunofluorescence microscope and the images were captured with magnifications of 100× and 200×. As shown in FIG. 3b, from the immunodetection of gD followed by secondary marking with FITC, it was possible to determine the in vitro expression of the vaccine antigen gDPOLYEP, seen by marking the cells in green in the viewed fields.

### Example 3—In Vivo Evaluation of the Vaccines' Immunogenicity

Animal experiments were conducted according to the Ethical Principles of Animal Experimentation established by the Ethics Committee in Animal Experimentation [Comitê de Ética em Experimentação Animal (CEUA)]. Mice of the C57BL/6 strain were inoculated intramuscularly according to the following immunization groups: 1) control plasmid (pCDNA3.1); 2) combined pgDNSA and pgDNSB plasmids; 3) polyepitope plasmid (pgDPOLYEP). Each group of animals (n=5-10) received two doses (50 ug/animal) of the vaccine formulations, with a 2-week interval between the doses being adopted. Immediately after the vaccines administration, the animals were electroporated at the application site, with 2 electric pulses of 45 V each being applied, with a duration interval of 450 ms (pulses that form pores in the cell membrane), and 4 pulses of 20 V each, with a duration of 450 ms (transfer pulses), using the NEPA21 SuperElectroporator equipment (NepaGeneCo., Ltd.; Chiba, Japan).

Intracellular cytokines (ICS) were detected in splenocytes of immunized mice 14 days after the administration of the last vaccine dose. To obtain splenocytes, the spleen of immunized mice was collected after euthanasia and macerated to obtain a cell suspension. The cells obtained were treated for 5 minutes on ice with Ack Lysing Buffer (BioSource International) until red blood cells rupture and then centrifuged at 1500 rpm for 5 minutes. After 2 washing cycles with RPMI medium, the splenocytes obtained were cultured at a ratio of 106 cells/well for 2 hours (37° C., 5% CO₂) in the presence of antigen-specific stimulation (peptides: pep4-VSFCYMHHM (SEQ ID NO: 28); pep5-VAYFNMVYM (SEQ ID NO: 160)) followed by the addition of Brefeldin A (GolgiPlug; BD Biosciences) and stimulation for additional 4 hours. The cells were incubated with RPMI medium (negative control) or in combination with PMA/ionomycin (positive control). After the stimulation period, the cells were washed with PBS (2×), followed by labeling with antibodies conjugated with fluorophore against CD3 and murine (clone 145-2C11, Tonbo), CD8a (clone 53-6.7, BioLegend), CD11a (clone M17/4, eBioscience), CD49d (clone R1-2, eBioscience), all used in dilution of 1:200. The cells were then fixed and permeated with Cytofix/Cytoperm (BD Bioscience) and stained with monoclonals conjugated with fluorophore against mouse IFNγ (clone XMG1.2, Tonbo) and TNF (clone MP6-XT22, eBioscience). Data was collected in a BD LSRFortessa™ equipment and analyzed using FlowJo software. The cytokine IFNγ secreted by splenocytes of immunized animals was detected using the ELISPOT technique. Briefly, the splenocytes of the immunized animals were stimulated in vitro analogously to that previously described, but without the addition of Brefeldin A.

In order to characterize the response of T cells induced after vaccination, spleen cells were collected and obtained using the techniques described above. As shown in FIG. 4, after two intramuscular doses of the pgDPOLYEP DNA vaccine (50 μg), the CD8⁺ T cells vaccine-specific response was evaluated by ICS assay using 2 peptides restricted to H2-Db, contained in the DNA vaccine. The number of spleen cells able to produce IFNγ was determined, as well as the IFNγ and TNF production frequencies by CD8⁺ T cells activated and experimented with antigen (CD49d⁺CD11a^{hi}CD8α^{lo}). Notably, the CD8⁺ T cells of mice that received the pgDPOLYEP DNA vaccine exhibited CD8⁺ T cells with a dominant Th1 phenotype once stimulated with pep, with significantly higher numbers of IFNγ-, TNF-αnd IL-2-producing cells, once compared to the other DNA vaccines containing the NSA and NSB portion or the pcDNA3.1 vector (FIG. 4a-f).

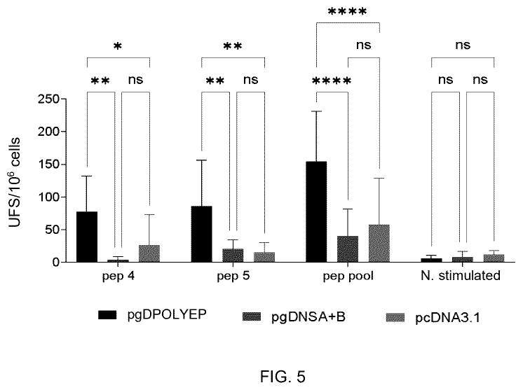

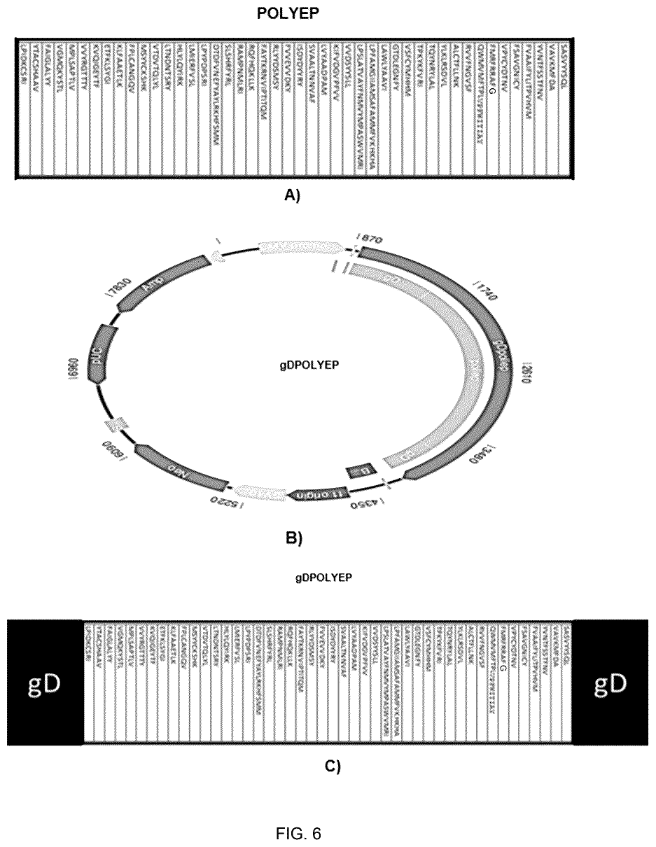

In order to detect IFNγ-producing cells, the spleen cells of the animals after two intramuscular doses of DNA vaccines were subjected to the ELISPOT assay (FIG. 5). As shown, the formulation composed of pgDPOLYEP induced the highest production of IFNγ when compared to the formulations where pgDNSA+pgDNSB or the empty vaccine vector pDNA3.1 was administered. Altogether, the results show that pgDPOLYEP is immunogenic capable of inducing cell-type immune response by inducing IFNγ- and TNF-producing CD8⁺ T lymphocytes.

Those skilled in the art will immediately understand that the invention is similarly applicable to other vaccine vehicles, that is, the use of the invention is not limited to the incorporation of the sequences encoding the fusion protein by a DNA-type vector. Examples include: the incorporation of the chimeric nucleotide sequence of the invention to vaccine viral vectors, such as those of adenovirus currently used (Janssen, Oxford) for SARS-COV-2 vaccines; the incorporation of the chimeric nucleotide sequence of the invention to microorganisms that express the fusion protein of the invention, for the subsequent inoculation of said preferably purified protein; the use of the chimeric RNA sequence of the invention with RNA vaccine vectors, such as those currently used (Moderna, Pfizer) for SARS-COV-2 vaccines.

The inventive concept now disclosed and exemplified in one or more ways was treated as an industrial secret and has not been previously disclosed until this patent application filing date or its priority. This industrial secret is immaterial assets of the applicant. The possible future publication of the patent application does not constitute, in itself, an authorization for use by third parties, being only useful as: (i) third-party acknowledgment of the existence of said industrial secret on the filing date; (ii) unequivocal indication of its holder; and (iii) stimulation to the development of new improvements from the concept now disclosed in order to avoid reinvestment in the development of the same asset already held by the applicant.

It is immediately warned that any commercial use requires the holder's authorization and that the unauthorized use entails sanctions provided for in the Law. In this context, it is clarified that from the disclosure of the present inventive concept, those skilled in the art may consider other ways of realizing the invention which are not identical to those merely exemplified above, but that in the event of commercial use intention, such forms may be considered as being within the scope of the attached claims.

1. A chimeric nucleotide sequence comprising the sequences encoding, in phase, a plurality of epitopes of a coronavirus non-structural protein.

2. The chimeric nucleotide sequence according to claim 1 comprising the sequences encoding multiple epitopes of replicase 1ab, aligned in phase.

3. The chimeric nucleotide sequence according to claim 1, wherein the nucleotide sequence encoding the polyepitope comprises a phased combination of the nucleotide sequences selected from SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91.

4. The chimeric nucleotide sequence according to claim 1, wherein the nucleotide sequence encoding the polyepitope is defined by SEQ ID NO: 95.

5. The chimeric nucleotide sequence according to claim 1, further comprising a nucleotide sequence that encodes a modified form of the glycoprotein D (gD) of herpes simplex type 1 (HSV-1).

6. The chimeric nucleotide sequence according to claim 1, wherein the nucleotide sequence encoding the modified form of the glycoprotein D (gD) of herpes simplex type 1 (HSV-1) is defined by SEQ ID NO: 97 and/or SEQ ID NO: 99.

7. The chimeric nucleotide sequence according to claim 1, further comprising a nucleotide sequence encoding a first spacer polypeptide.

8. The chimeric nucleotide sequence according to claim 1, further comprising:
a) a nucleotide sequence that encodes a first peptide comprising epitopes found in the amino acid sequence of the replicase polyprotein 1ab (PR1ab);
b) a nucleotide sequence encoding a first spacer polypeptide; and
c) a nucleotide sequence that encodes a modified form of the glycoprotein D (gD) of herpes simplex type 1 (HSV-1).

9. The chimeric nucleotide sequence according to claim 1, wherein the chimeric nucleotide sequence is defined by SEQ ID NO: 101.

10. A mammalian expression vector comprising
a chimeric nucleotide sequence encoding the polyepitope, as defined in claim 1; and
one or more expression promoters functionally linked to said chimeric nucleotide sequence.

11. An RNA vaccine comprising an RNA sequence that encodes a plurality of epitopes of a coronavirus non-structural protein.

12. The RNA vaccine according to claim 11, further comprising the sequences encoding multiple epitopes of replicase 1ab, aligned in phase.

13. The RNA vaccine according to claim 11 comprising a combination of the RNA sequences selected from SEQ ID NO: 113 to SEQ ID NO: 158.

14. A chimeric fusion protein comprising a plurality of epitopes of a coronavirus non-structural protein.

15. The chimeric fusion protein according to claim 14, comprising a combination of the peptide sequences of replicase 1ab selected from SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92.

16. The chimeric fusion protein according to claim 14 comprising SEQ ID NO: 96.

17. The chimeric fusion protein according to claim 14, further comprising a polypeptide sequence of a modified form of the glycoprotein D (gD) of herpes simplex type 1 (HSV-1).

18. The chimeric fusion protein according to claim 17, wherein the polypeptide sequence of the modified form of the glycoprotein D (gD) of herpes simplex type 1 (HSV-1) is defined by SEQ ID NO: 98 and/or SEQ ID NO: 100.

19. The chimeric fusion protein according to claim 14, further comprising a sequence of a first spacer polypeptide.

20. The chimeric fusion protein according to claim 14, comprising:
a) a first peptide consisting of epitopes found in the amino acid sequence of the replicase polyprotein 1ab (PR1ab);
b) a first spacer; and
c) modified form of the glycoprotein D (gD) of herpes simplex type 1 (HSV-1).

21. The chimeric fusion protein according to claim 14, comprising the amino acid sequence defined by SEQ ID NO: 108.

22. A method for the preparation of a vaccine for coronavirus, SARS-COV-2 and/or related viruses comprising a chimeric nucleotide sequence as defined in claim 1 or a chimeric fusion protein as defined in claim 14.

23. A method of preparation of a vaccine for coronavirus, SARS-COV-2, and/or related viruses, wherein the method comprises preparation of a chimeric nucleotide sequence as defined in claim 1 or a chimeric fusion protein as defined in claim 14.

24. A method of inducing cell-type immune response comprising administering an effective dosage of a chimeric nucleotide sequence as defined in claim 1 or a chimeric fusion protein as defined in claim 14.

25. A method of treatment of viral diseases comprising administering an effective dosage of a chimeric nucleotide sequence as defined in claim 1 or a chimeric fusion protein as defined in claim 14.


SEQUENCE LISTING (160 sequences)

SEQ ID NO: 1 - DNA, 27 bp, Sars-CoV-2
agcgccagcgtgtactacagccagctg

SEQ ID NO: 2 - protein, 9 aa, Sars-CoV-2
SASVYYSQL

SEQ ID NO: 3 - DNA, 24 bp, Sars-CoV-2
gtggccgtgaagatgttcgacgcc

SEQ ID NO: 4 - protein, 8 aa, Sars-CoV-2
VAVKMFDA

SEQ ID NO: 5 - DNA, 33 bp, Sars-CoV-2
tacgtgaacaccttcagcagcaccttcaacgtg

SEQ ID NO: 6 - protein, 11 aa, Sars-CoV-2
YVNTFSSTFNV

SEQ ID NO: 7 - DNA, 45 bp, Sars-CoV-2
ttcgtggccgccatcttctacctgatcacccccgtgcacgtgatg

SEQ ID NO: 8 - protein, 15 aa, Sars-CoV-2
FVAAIFYLITPVHVM

SEQ ID NO: 9 - DNA, 27 bp, Sars-CoV-2
ttcagcgccgtgggcaacatctgctac

SEQ ID NO: 10 - protein, 9 aa, Sars-CoV-2
FSAVGNICY

SEQ ID NO: 11 - DNA, 27 bp, Sars-CoV-2
gtgccctactgctacgacaccaacgtg

SEQ ID NO: 12 - protein, 9 aa, Sars-CoV-2
VPYCYDTNV

SEQ ID NO: 13 - DNA, 27 bp, Sars-CoV-2
ttcatgaggttcaggagggccttcggc

SEQ ID NO: 14 - protein, 9 aa, Sars-CoV-2
FMRFRRAFG

SEQ ID NO: 15 - DNA, 54 bp, Sars-CoV-2
cagtggatggtgatgttcacccccctggtgcccttctggatcaccatcgcctac

SEQ ID NO: 16 - protein, 18 aa, Sars-CoV-2
QWMVMFTPLVPFWITIAY

SEQ ID NO: 17 - DNA, 27 bp, Sars-CoV-2
agggtggtgttcaacggcgtgagcttc

SEQ ID NO: 18 - protein, 9 aa, Sars-CoV-2
RVVFNGVSF

SEQ ID NO: 19 - DNA, 27 bp, Sars-CoV-2
gccctgtgcaccttcctgctgaacaag

SEQ ID NO: 20 - protein, 9 aa, Sars-CoV-2
ALCTFLLNK

SEQ ID NO: 21 - DNA, 27 bp, Sars-CoV-2
tacctgaagctgaggagcgacgtgctg

SEQ ID NO: 22 - protein, 9 aa, Sars-CoV-2
YLKLRSDVL

SEQ ID NO: 23 - DNA, 27 bp, Sars-CoV-2
acccagtacaacaggtacctggccctg

SEQ ID NO: 24 - protein, 9 aa, Sars-CoV-2
TQYNRYLAL

SEQ ID NO: 25 - DNA, 26 bp, Sars-CoV-2
cccccaagtacaagttcgtgaggatc

SEQ ID NO: 26 - protein, 9 aa, Sars-CoV-2
TPKYKFVRI

SEQ ID NO: 27 - DNA, 27 bp, Sars-CoV-2
gtgagcttctgctacatgcaccacatg

SEQ ID NO: 28 - protein, 9 aa, Sars-CoV-2
VSFCYMHHM

SEQ ID NO: 29 - DNA, 27 bp, Sars-CoV-2
ggcaccgacctggagggcaacttctac

SEQ ID NO: 30 - protein, 9 aa, Sars-CoV-2
GTDLEGNFY

SEQ ID NO: 31 - DNA, 27 bp, Sars-CoV-2
ctggcctggctgtacgccgccgtgatc

SEQ ID NO: 32 - protein, 9 aa, Sars-CoV-2
LAWLYAAVI

SEQ ID NO: 33 - DNA, 69 bp, Sars-CoV-2
ctgcccttcgccatgggcatcatcgccatgagcgccttcgccatgatgttcgtgaagcacaagcacgcc

SEQ ID NO: 34 - protein, 23 aa, Sars-CoV-2
LPFAMGIIAMSAFAMMFVKHKHA

SEQ ID NO: 35 - DNA, 69 bp, Sars-CoV-2
ctgcccagcctggccaccgtggcctacttcaacatggtgtacatgcccgccagctgggtgatgaggatc

SEQ ID NO: 36 - protein, 23 aa, Sars-CoV-2
LPSLATVAYFNMVYMPASWVMRI

SEQ ID NO: 37 - DNA, 27 bp, Sars-CoV-2
gtggtggacagctactacagcctgctg

SEQ ID NO: 38 - protein, 9 aa, Sars-CoV-2
VVDSYYSLL

SEQ ID NO: 39 - DNA, 33 bp, Sars-CoV-2
aagatcttcgtggacggcgtgcccttcgtggtg

SEQ ID NO: 40 - protein, 11 aa, Sars-CoV-2
KIFVDGVPFVV

SEQ ID NO: 41 - DNA, 27 bp, Sars-CoV-2
ctggtgtacgccgccgaccccgccatg

SEQ ID NO: 42 - protein, 9 aa, Sars-CoV-2
LVYAADPAM

SEQ ID NO: 43 - DNA, 33 bp, Sars-CoV-2
agcgtggccgccctgaccaacaacgtggccttc

SEQ ID NO: 44 - protein, 11 aa, Sars-CoV-2
SVAALTNNVAF

SEQ ID NO: 45 - DNA, 27 bp, Sars-CoV-2
atcagcgactacgactactacaggtac

SEQ ID NO: 46 - protein, 9 aa, Sars-CoV-2
ISDYDYYRY

SEQ ID NO: 47 - DNA, 27 bp, Sars-CoV-2
ttcgtggtggaggtggtggacaagtac

SEQ ID NO: 48 - protein, 9 aa, Sars-CoV-2
FVVEVVDKY

SEQ ID NO: 49 - DNA, 27 bp, Sars-CoV-2
aggctgtactacgacagcatgagctac

SEQ ID NO: 50 - protein, 9 aa, Sars-CoV-2
RLYYDSMSY

SEQ ID NO: 51 - DNA, 45 bp, Sars-CoV-2
ttcgcctacaccaagaggaacgtgatccccaccatcacccagatg

SEQ ID NO: 52 - protein, 15 aa, Sars-CoV-2
FAYTKRNVIPTITQM

SEQ ID NO: 53 - DNA, 27 bp, Sars-CoV-2
aggcagttccaccagaagctgctgaag

SEQ ID NO: 54 - protein, 9 aa, Sars-CoV-2
RQFHQKLLK

SEQ ID NO: 55 - DNA, 27 bp, Sars-CoV-2
agggccatgcccaacatgctgaggatc

SEQ ID NO: 56 - protein, 9 aa, Sars-CoV-2
RAMPNMLRI

SEQ ID NO: 57 - DNA, 27 bp, Sars-CoV-2
agcctgagccacaggttctacaggctg

SEQ ID NO: 58 - protein, 9 aa, Sars-CoV-2
SLSHRFYRL

SEQ ID NO: 59 - DNA, 57 bp, Sars-CoV-2
gacaccgacttcgtgaacgagttctacgcctacctgaggaagcacttcagcatgatg

SEQ ID NO: 60 - protein, 19 aa, Sars-CoV-2
DTDFVNEFYAYLRKHFSMM

SEQ ID NO: 61 - DNA, 27 bp, Sars-CoV-2
ctgccctaccccgaccccagcaggatc

SEQ ID NO: 62 - protein, 9 aa, Sars-CoV-2
LPYPDPSRI

SEQ ID NO: 63 - DNA, 27 bp, Sars-CoV-2
ctgatgatcgagaggttcgtgagcctg

SEQ ID NO: 64 - protein, 9 aa, Sars-CoV-2
LMIERFVSL

SEQ ID NO: 65 - DNA, 27 bp, Sars-CoV-2
cacctgtacctgcagtacatcaggaag

SEQ ID NO: 66 - protein, 9 aa, Sars-CoV-2
HLYLQYIRK

SEQ ID NO: 67 - DNA, 27 bp, Sars-CoV-2
ctgaccaacgacaacaccagcaggtac

SEQ ID NO: 68 - protein, 9 aa, Sars-CoV-2
LTNDNTSRY

SEQ ID NO: 69 - DNA, 27 bp, Sars-CoV-2
gtgaccgacgtgacccagctgtacctg

SEQ ID NO: 70 - protein, 9 aa, Sars-CoV-2
VTDVTQLYL

SEQ ID NO: 71 - DNA, 27 bp, Sars-CoV-2
atgagctactactgcaagagccacaag

SEQ ID NO: 72 - protein, 9 aa, Sars-CoV-2
MSYYCKSHK

SEQ ID NO: 73 - DNA, 27 bp, Sars-CoV-2
ttccccctgtgcgccaacggccaggtg

SEQ ID NO: 74 - protein, 9 aa, Sars-CoV-2
FPLCANGQV

SEQ ID NO: 75 - DNA, 27 bp, Sars-CoV-2
aagctgttcgccgccgagaccctgaag

SEQ ID NO: 76 - protein, 9 aa, Sars-CoV-2
KLFAAETLK

SEQ ID NO: 77 - DNA, 27 bp, Sars-CoV-2
gagaccttcaagctgagctacggcatc

SEQ ID NO: 78 - protein, 9 aa, Sars-CoV-2
ETFKLSYGI

SEQ ID NO: 79 - DNA, 27 bp, Sars-CoV-2
aaggtgcagatcggcgagtacaccttc

SEQ ID NO: 80 - protein, 9 aa, Sars-CoV-2
KVQIGEYTF

SEQ ID NO: 81 - DNA, 27 bp, Sars-CoV-2
gtggtgtacaggggcaccaccacctac

SEQ ID NO: 82 - protein, 9 aa, Sars-CoV-2
VVYRGTTTY

SEQ ID NO: 83 - DNA, 27 bp, Sars-CoV-2
atgcccctgagcgcccccaccctggtg

SEQ ID NO: 84 - protein, 9 aa, Sars-CoV-2
MPLSAPTLV

SEQ ID NO: 85 - DNA, 27 bp, Sars-CoV-2
gtgggcatgcagaagtacagcaccctg

SEQ ID NO: 86 - protein, 9 aa, Sars-CoV-2
VGMQKYSTL

SEQ ID NO: 87 - DNA, 27 bp, Sars-CoV-2
ttcgccatcggcctggccctgtactac

SEQ ID NO: 88 - protein, 9 aa, Sars-CoV-2
FAIGLALYY

SEQ ID NO: 89 - DNA, 27 bp, Sars-CoV-2
tacaccgcctgcagccacgccgccgtg

SEQ ID NO: 90 - protein, 9 aa, Sars-CoV-2
YTACSHAAV

SEQ ID NO: 91 - DNA, 27 bp, Sars-CoV-2
ctgcccatcgacaagtgcagcaggatc

SEQ ID NO: 92 - protein, 9 aa, Sars-CoV-2
LPIDKCSRI

SEQ ID NO: 93 - DNA, 12 bp, Artificial Sequence
  Spacer coding nucleotide sequence
ggcggcggcagc

SEQ ID NO: 94 - protein, 4 aa, Artificial Sequence
  Spacer coding peptide sequence
GGGS

SEQ ID NO: 95 - DNA, 1974 bp, Artificial Sequence
  Nucleotide sequence coding for the polyepitope
agcgccagcgtgtactacagccagctgggcggcggcagcgtggccgtgaagatgttcgacgccggcggcggcagctacgtgaacaccttcagcagcaccttcaacgtgggcggcggcagcttcgtggccgccatcttctacctgatcacccccgtgcacgtgatgggcggcggcagcttcagcgccgtgggcaacatctgctacggcggcggcagcgtgccctactgctacgacaccaacgtgggcggcggcagcttcatgaggttcaggagggccttcggcggcggcggcagccagtggatggtgatgttcacccccctggtgcccttctggatcaccatcgcctacggcggcggcagcagggtggtgttcaacggcgtgagcttcggcggcggcagcgccctgtgcaccttcctgctgaacaagggcggcggcagctacctgaagctgaggagcgacgtgctgggcggcggcagcacccagtacaacaggtacctggccctgggcggcggcagcacccccaagtacaagttcgtgaggatcggcggcggcagcgtgagcttctgctacatgcaccacatgggcggcggcagcggcaccgacctggagggcaacttctacggcggcggcagcctggcctggctgtacgccgccgtgatcggcggcggcagcctgcccttcgccatgggcatcatcgccatgagcgccttcgccatgatgttcgtgaagcacaagcacgccggcggcggcagcctgcccagcctggccaccgtggcctacttcaacatggtgtacatgcccgccagctgggtgatgaggatcggcggcggcagcgtggtggacagctactacagcctgctgggcggcggcagcaagatcttcgtggacggcgtgcccttcgtggtgggcggcggcagcctggtgtacgccgccgaccccgccatgggcggcggcagcagcgtggccgccctgaccaacaacgtggccttcggcggcggcagcatcagcgactacgactactacaggtacggcggcggcagcttcgtggtggaggtggtggacaagtacggcggcggcagcaggctgtactacgacagcatgagctacggcggcggcagcttcgcctacaccaagaggaacgtgatccccaccatcacccagatgggcggcggcagcaggcagttccaccagaagctgctgaagggcggcggcagcagggccatgcccaacatgctgaggatcggcggcggcagcagcctgagccacaggttctacaggctgggcggcggcagcgacaccgacttcgtgaacgagttctacgcctacctgaggaagcacttcagcatgatgggcggcggcagcctgccctaccccgaccccagcaggatcggcggcggcagcctgatgatcgagaggttcgtgagcctgggcggcggcagccacctgtacctgcagtacatcaggaagggcggcggcagcctgaccaacgacaacaccagcaggtacggcggcggcagcgtgaccgacgtgacccagctgtacctgggcggcggcagcatgagctactactgcaagagccacaagggcggcggcagcttccccctgtgcgccaacggccaggtgggcggcggcagcaagctgttcgccgccgagaccctgaagggcggcggcagcgagaccttcaagctgagctacggcatcggcggcggcagcaaggtgcagatcggcgagtacaccttcggcggcggcagcgtggtgtacaggggcaccaccacctacggcggcggcagcatgcccctgagcgcccccaccctggtgggcggcggcagcgtgggcatgcagaagtacagcaccctgggcggcggcagcttcgccatcggcctggccctgtactacggcggcggcagctacaccgcctgcagccacgccgccgtgggcggcggcagcctgcccatcgacaagtgcagcaggatc

SEQ ID NO: 96 - protein, 658 aa, Artificial Sequence
  Peptide sequence encoding the polyepitope
SASVYYSQLGGGSVAVKMFDAGGGSYVNTFSSTFNVGGGSFVAAIFYLITPVHVMGGGSFSAVGNICYGGGSVPYCYDTNVGGGSFMRFRRAFGGGGSQWMVMFTPLVPFWITIAYGGGSRVVFNGVSFGGGSALCTFLLNKGGGSYLKLRSDVLGGGSTQYNRYLALGGGSTPKYKFVRIGGGSVSFCYMHHMGGGSGTDLEGNFYGGGSLAWLYAAVIGGGSLPFAMGIIAMSAFAMMFVKHKHAGGGSLPSLATVAYFNMVYMPASWVMRIGGGSVVDSYYSLLGGGSKIFVDGVPFVVGGGSLVYAADPAMGGGSSVAALTNNVAFGGGSISDYDYYRYGGGSFVVEVVDKYGGGSRLYYDSMSYGGGSFAYTKRNVIPTITQMGGGSRQFHQKLLKGGGSRAMPNMLRIGGGSSLSHRFYRLGGGSDTDFVNEFYAYLRKHFSMMGGGSLPYPDPSRIGGGSLMIERFVSLGGGSHLYLQYIRKGGGSLTNDNTSRYGGGSVTDVTQLYLGGGSMSYYCKSHKGGGSFPLCANGQVGGGSKLFAAETLKGGGSETFKLSYGIGGGSKVQIGEYTFGGGSVVYRGTTTYGGGSMPLSAPTLVGGGSVGMQKYSTLGGGSFAIGLALYYGGGSYTACSHAAVGGGSLPIDKCSRI

SEQ ID NO: 97 - DNA, 819 bp, Artificial Sequence
  Nucleotide sequence coding for the modified form of herpes simplex type-1 (HSV-1) glycoprotein D (gD) - gD fragment in the N-terminal portion
aagcttaccatgatgggaggggctgctgccagactgggagctgtgattctgttcgtggtcattgtggggctgcatggagtgcgggggaaatacgctctggccgatgcaagcctgaaaatggcagaccctaacagattcagagggaaagacctgcccgtgctggatcagctgactgatcccccaggcgtgagacgggtgtatcatatccaagctggcctgccagatccattccagcccccctccctgcctatcaccgtctactacgcagtgctggagagagcctgtaggtccgtgctgctgaatgctcctagcgaagcccctcaaattgtgcggggcgcctccgaagacgtgagaaagcagccatataatctgaccatcgcttggtttaggatgggcggcaactgcgccattcctatcaccgtgatggagtatactgaatgcagctataacaagtccctgggcgcctgcccaattagaacccaacctaggtggaactattatgacagcttttccgcagtcagcgaagacaacctggggttcctgatgcacgcccccgcttttgaaactgccgggacatatctgcggctggtcaaaatcaacgattggacagaaatcacccagttcatcctggaacatagggccaaagggagctgcaaatatgccctgccactgaggatccccccttccgcttgtctgtcccctcaagcctaccagcaaggagtgacagtggacagcatcggcatgctgcccagattcattcccgagaaccagagaactgtggctgtgtactccctgaagattgccggatggcacggatcc

SEQ ID NO: 98 - protein, 270 aa, Artificial Sequence
  Amino acid sequence of the modified form of herpes simplex type-1 (HSV-1) gD - gD fragment in the N-terminal portion
MMGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGS

SEQ ID NO: 99 - DNA, 396 bp, Artificial Sequence
  Nucleotide sequence coding for the modified form of herpes simplex type-1 (HSV-1) glycoprotein D (gD) - gD fragment in the C-terminal portion
ggatcctctagaaaggccccttacacttccacactgctgccccctgagctgagcgaaacacccaatgccacccagcccgagctggctcccgaggaccccgaggacagcgccctgctggaggacccagtcggcacagtcgccccacagatccctcccaattggcacattcccagcattcaggacgcagcaaccccctaccacccaccagcaacccctaataatatgggcctgatcgcaggcgcagtcggaggctccctgctggcagcactggtcatctgtgggatcgtctactggatgaggcggcggacacagaaggcaccaaagcggattcggctgccacacattcgggaagacgatcagccttcctcccaccagccactgttttactaactcgag

SEQ ID NO: 100 - protein, 129 aa, Artificial Sequence
  Amino acid sequence encoding the modified form of herpes simplex type-1 (HSV-1) glycoprotein D (gD) - gD fragment in the C-terminal portion
GSSRKAPYTSTLLPPELSETPNATQPELAPEDPEDSALLEDPVGTVAPQIPPNWHIPSIQDAATPYHPPATPNNMGLIAGAVGGSLLAALVICGIVYWMRRRTQKAPKRIRLPHIREDDQPSSHQPLFY

SEQ ID NO: 101 - DNA, 3189 bp, Artificial Sequence
  Covid19_gD_POLIEP (DNA)  (5′-->3′)
aagcttaccatgatgggaggggctgctgccagactgggagctgtgattctgttcgtggtcattgtggggctgcatggagtgcgggggaaatacgctctggccgatgcaagcctgaaaatggcagaccctaacagattcagagggaaagacctgcccgtgctggatcagctgactgatcccccaggcgtgagacgggtgtatcatatccaagctggcctgccagatccattccagcccccctccctgcctatcaccgtctactacgcagtgctggagagagcctgtaggtccgtgctgctgaatgctcctagcgaagcccctcaaattgtgcggggcgcctccgaagacgtgagaaagcagccatataatctgaccatcgcttggtttaggatgggcggcaactgcgccattcctatcaccgtgatggagtatactgaatgcagctataacaagtccctgggcgcctgcccaattagaacccaacctaggtggaactattatgacagcttttccgcagtcagcgaagacaacctggggttcctgatgcacgcccccgcttttgaaactgccgggacatatctgcggctggtcaaaatcaacgattggacagaaatcacccagttcatcctggaacatagggccaaagggagctgcaaatatgccctgccactgaggatccccccttccgcttgtctgtcccctcaagcctaccagcaaggagtgacagtggacagcatcggcatgctgcccagattcattcccgagaaccagagaactgtggctgtgtactccctgaagattgccggatggcacggatccagcgccagcgtgtactacagccagctgggcggcggcagcgtggccgtgaagatgttcgacgccggcggcggcagctacgtgaacaccttcagcagcaccttcaacgtgggcggcggcagcttcgtggccgccatcttctacctgatcacccccgtgcacgtgatgggcggcggcagcttcagcgccgtgggcaacatctgctacggcggcggcagcgtgccctactgctacgacaccaacgtgggcggcggcagcttcatgaggttcaggagggccttcggcggcggcggcagccagtggatggtgatgttcacccccctggtgcccttctggatcaccatcgcctacggcggcggcagcagggtggtgttcaacggcgtgagcttcggcggcggcagcgccctgtgcaccttcctgctgaacaagggcggcggcagctacctgaagctgaggagcgacgtgctgggcggcggcagcacccagtacaacaggtacctggccctgggcggcggcagcacccccaagtacaagttcgtgaggatcggcggcggcagcgtgagcttctgctacatgcaccacatgggcggcggcagcggcaccgacctggagggcaacttctacggcggcggcagcctggcctggctgtacgccgccgtgatcggcggcggcagcctgcccttcgccatgggcatcatcgccatgagcgccttcgccatgatgttcgtgaagcacaagcacgccggcggcggcagcctgcccagcctggccaccgtggcctacttcaacatggtgtacatgcccgccagctgggtgatgaggatcggcggcggcagcgtggtggacagctactacagcctgctgggcggcggcagcaagatcttcgtggacggcgtgcccttcgtggtgggcggcggcagcctggtgtacgccgccgaccccgccatgggcggcggcagcagcgtggccgccctgaccaacaacgtggccttcggcggcggcagcatcagcgactacgactactacaggtacggcggcggcagcttcgtggtggaggtggtggacaagtacggcggcggcagcaggctgtactacgacagcatgagctacggcggcggcagcttcgcctacaccaagaggaacgtgatccccaccatcacccagatgggcggcggcagcaggcagttccaccagaagctgctgaagggcggcggcagcagggccatgcccaacatgctgaggatcggcggcggcagcagcctgagccacaggttctacaggctgggcggcggcagcgacaccgacttcgtgaacgagttctacgcctacctgaggaagcacttcagcatgatgggcggcggcagcctgccctaccccgaccccagcaggatcggcggcggcagcctgatgatcgagaggttcgtgagcctgggcggcggcagccacctgtacctgcagtacatcaggaagggcggcggcagcctgaccaacgacaacaccagcaggtacggcggcggcagcgtgaccgacgtgacccagctgtacctgggcggcggcagcatgagctactactgcaagagccacaagggcggcggcagcttccccctgtgcgccaacggccaggtgggcggcggcagcaagctgttcgccgccgagaccctgaagggcggcggcagcgagaccttcaagctgagctacggcatcggcggcggcagcaaggtgcagatcggcgagtacaccttcggcggcggcagcgtggtgtacaggggcaccaccacctacggcggcggcagcatgcccctgagcgcccccaccctggtgggcggcggcagcgtgggcatgcagaagtacagcaccctgggcggcggcagcttcgccatcggcctggccctgtactacggcggcggcagctacaccgcctgcagccacgccgccgtgggcggcggcagcctgcccatcgacaagtgcagcaggatcggatcctctagaaaggccccttacacttccacactgctgccccctgagctgagcgaaacacccaatgccacccagcccgagctggctcccgaggaccccgaggacagcgccctgctggaggacccagtcggcacagtcgccccacagatccctcccaattggcacattcccagcattcaggacgcagcaaccccctaccacccaccagcaacccctaataatatgggcctgatcgcaggcgcagtcggaggctccctgctggcagcactggtcatctgtgggatcgtctactggatgaggcggcggacacagaaggcaccaaagcggattcggctgccacacattcgggaagacgatcagccttcctcccaccagccactgttttactaactcgag

SEQ ID NO: 102 - protein, 1057 aa, Artificial Sequence
  Covid19_gD_POLIEP (protein)
MMGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGSSASVYYSQLGGGSVAVKMFDAGGGSYVNTFSSTFNVGGGSFVAAIFYLITPVHVMGGGSFSAVGNICYGGGSVPYCYDTNVGGGSFMRFRRAFGGGGSQWMVMFTPLVPFWITIAYGGGSRVVFNGVSFGGGSALCTFLLNKGGGSYLKLRSDVLGGGSTQYNRYLALGGGSTPKYKFVRIGGGSVSFCYMHHMGGGSGTDLEGNFYGGGSLAWLYAAVIGGGSLPFAMGIIAMSAFAMMFVKHKHAGGGSLPSLATVAYFNMVYMPASWVMRIGGGSVVDSYYSLLGGGSKIFVDGVPFVVGGGSLVYAADPAMGGGSSVAALTNNVAFGGGSISDYDYYRYGGGSFVVEVVDKYGGGSRLYYDSMSYGGGSFAYTKRNVIPTITQMGGGSRQFHQKLLKGGGSRAMPNMLRIGGGSSLSHRFYRLGGGSDTDFVNEFYAYLRKHFSMMGGGSLPYPDPSRIGGGSLMIERFVSLGGGSHLYLQYIRKGGGSLTNDNTSRYGGGSVTDVTQLYLGGGSMSYYCKSHKGGGSFPLCANGQVGGGSKLFAAETLKGGGSETFKLSYGIGGGSKVQIGEYTFGGGSVVYRGTTTYGGGSMPLSAPTLVGGGSVGMQKYSTLGGGSFAIGLALYYGGGSYTACSHAAVGGGSLPIDKCSRIGSSRKAPYTSTLLPPELSETPNATQPELAPEDPEDSALLEDPVGTVAPQIPPNWHIPSIQDAATPYHPPATPNNMGLIAGAVGGSLLAALVICGIVYWMRRRTQKAPKRIRLPHIREDDQPSSHQPLFY

SEQ ID NO: 103 - DNA, 4548 bp, Artificial Sequence
  Covid19_gD_NSa (DNA) (5′-->3′)
aagcttaccatgatgggaggggctgctgccagactgggagctgtgattctgttcgtggtcattgtggggctgcatggagtgcgggggaaatacgctctggccgatgcaagcctgaaaatggcagaccctaacagattcagagggaaagacctgcccgtgctggatcagctgactgatcccccaggcgtgagacgggtgtatcatatccaagctggcctgccagatccattccagcccccctccctgcctatcaccgtctactacgcagtgctggagagagcctgtaggtccgtgctgctgaatgctcctagcgaagcccctcaaattgtgcggggcgcctccgaagacgtgagaaagcagccatataatctgaccatcgcttggtttaggatgggcggcaactgcgccattcctatcaccgtgatggagtatactgaatgcagctataacaagtccctgggcgcctgcccaattagaacccaacctaggtggaactattatgacagcttttccgcagtcagcgaagacaacctggggttcctgatgcacgcccccgcttttgaaactgccgggacatatctgcggctggtcaaaatcaacgattggacagaaatcacccagttcatcctggaacatagggccaaagggagctgcaaatatgccctgccactgaggatccccccttccgcttgtctgtcccctcaagcctaccagcaaggagtgacagtggacagcatcggcatgctgcccagattcattcccgagaaccagagaactgtggctgtgtactccctgaagattgccggatggcacggatccagcgcgaaaagcgcgagcgtgtattatagccagctgatgtgccagccgattctgctgctggatcaggcgctggtgagcgatgtgggcgatagcgcggaagtggcggtgaaaatgtttgatgcgtatgtgaacacctttagcagcacctttaacgtgccgatggaaaaactgaaaaccctggtggcgaccgcggaagcggaactggcgaaaaacgtgagcctggataacgtgctgagcacctttattagcgcggcgcgccagggctttgtggatagcgatgtggaaaccaaagatgtggtggaatgcctgaaactgagccatcagagcgatattgaagtgaccggcgatagctgcaacaactatatgctgacctataacaaagtggaaaacatgaccccgcgcgatctgggcgcgtgcattgattgcagcgcgcgccatattaacgcgcaggtggcgaaaagccataacattgcgctgatttggaacgtgaaagattttatgagcctgagcgaacagctgcgcaaacagattcgcagcgcggcgaaaaaaaacaacctgccgtttaaactgacctgcgcgaccacccgccaggtggtgaacgtggtgaccaccaaaattgcgctgaaaggcggcaaaattgtgaacaactggctgaaacagctgattaaagtgaccctggtgtttctgtttgtggcggcgattttttatctgattaccccggtgcatgtgatgagcaaacataccgattttagcagcgaaattattggctataaagcgattgatggcggcgtgacccgcgatattgcgagcaccgatacctgctttgcgaacaaacatgcggattttgatacctggtttagccagcgcggcggcagctataccaacgataaagcgtgcccgctgattgcggcggtgattacccgcgaagtgggctttgtggtgccgggcctgccgggcaccattctgcgcaccaccaacggcgattttctgcattttctgccgcgcgtgtttagcgcggtgggcaacatttgctataccccgagcaaactgattgaatataccgattttgcgaccagcgcgtgcgtgctggcggcggaatgcaccatttttaaagatgcgagcggcaaaccggtgccgtattgctatgataccaacgtgctggaaggcagcgtggcgtatgaaagcctgcgcccggatacccgctatgtgctgatggatggcagcattattcagtttccgaacacctatctggaaggcagcgtgcgcgtggtgaccacctttgatagcgaatattgccgccatggcacctgcgaacgcagcgaagcgggcgtgtgcgtgagcaccagcggccgctgggtgctgaacaacgattattatcgcagcctgccgggcgtgttttgcggcgtggatgcggtgaacctgctgaccaacatgtttaccccgctgattcagccgattggcgcgctggatattagcgcgagcattgtggcgggcggcattgtggcgattgtggtgacctgcctggcgtattattttatgcgctttcgccgcgcgtttggcgaatatagccatgtggtggcgtttaacaccctgctgtttctgatgagctttaccgtgctgtgcctgaccccggtgtatagctttctgccgggcgtgtatagcgtgatttatctgtatctgaccttttatctgaccaacgatgtgagctttctggcgcatattcagtggatggtgatgtttaccccgctggtgccgttttggattaccattgcgtatattatttgcattagcaccaaacatttttattggttttttagcaactatctgaaacgccgcgtggtgtttaacggcgtgagctttagcacctttgaagaagcggcgctgtgcacctttctgctgaacaaagaaatgtatctgaaactgcgcagcgatgtgctgctgccgctgacccagtataaccgctatctggcgctgtataacaaatataaatattttagcggcgcgatggataccaccagctatcgcgaagcggcgtgctgccatctggcgaaagcgctgaacgattttagcaacagcggcagcgatgtgctgtatcagccgccgcagaccagcattaccagcgcggtgctgcagagcggctttcgcaaaatggcgtttccgagcggcaaagtggaaggctgcatggtgcaggtgacctgcggcaccaccaccctgaacggcctgtggctggatgatgtggtgtattgcccgcgccatgtgatttgcaccagcgaagatatgctgaacccgaactatgaagatctgctgattcgcaaaagcaaccataactttctggtgcaggcgggcaacgtgcagctgcgcgtgattggccatagcatgcagaactgcgtgctgaaactgaaagtggataccgcgaacccgaaaaccccgaaatataaatttgtgcgcattcagccgggccagacctttagcgtgctggcgtgctataacggcagcccgagcggcgtgtatcagtgcgcgatgcgcccgaactttaccattaaaggcagctttctgaacggcagctgcggcagcgtgggctttaacattgattatgattgcgtgagcttttgctatatgcatcatatggaactgccgaccggcgtgcatgcgggcaccgatctggaaggcaacttttatggcccgtttgtggatcgccagaccgcgcaggcggcgggcaccgataccaccattaccgtgaacgtgctggcgtggctgtatgcggcggtgattaacggcgatcgctggtttctgaaccgctttaccaccaccctgaacgattttaacctggtggcgatgaaatataactatgaaccgctgacccaggatcatgtggatattctgggcccgctgagcgcgcagaccggcattgcggtgctggatatgtgcgcgagcctgaaagaactgctgcagaacggcatgaacggccgcaccattctgggcagcgcgctgctggaagatgaatttaccccgtttgatgtggtgcgccagtgcagcggcgtgacctttcagagcgcggtgaaacgcaccattaaaggcacccatcattggctgctgctgaccattctgaccagcctgctggtgctggtgcagagcacccagtggagcctgtttttttttttttatgaaaacgcgtttctgccgtttgcgatgggcattattgcgatgagcgcgtttgcgatgatgtttgtgaaacataaacatgcgtttctgtgcctgtttctgctgccgagcctggcgaccgtggcgtattttaacatggtgtatatgccggcgagctgggtgatgcgcattggatcctctagaaaggccccttacacttccacactgctgccccctgagctgagcgaaacacccaatgccacccagcccgagctggctcccgaggaccccgaggacagcgccctgctggaggacccagtcggcacagtcgccccacagatccctcccaattggcacattcccagcattcaggacgcagcaaccccctaccacccaccagcaacccctaataatatgggcctgatcgcaggcgcagtcggaggctccctgctggcagcactggtcatctgtgggatcgtctactggatgaggcggcggacacagaaggcaccaaagcggattcggctgccacacattcgggaagacgatcagccttcctcccaccagccactgttttactaactcgag

SEQ ID NO: 104 - protein, 1513 aa, Artificial Sequence
  Covid19_gD_NSa (protein)
KLTMMGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGSSAKSASVYYSQLMCQPILLLDQALVSDVGDSAEVAVKMFDAYVNTFSSTFNVPMEKLKTLVATAEAELAKNVSLDNVLSTFISAARQGFVDSDVETKDVVECLKLSHQSDIEVTGDSCNNYMLTYNKVENMTPRDLGACIDCSARHINAQVAKSHNIALIWNVKDFMSLSEQLRKQIRSAAKKNNLPFKLTCATTRQVVNVVTTKIALKGGKIVNNWLKQLIKVTLVFLFVAAIFYLITPVHVMSKHTDFSSEIIGYKAIDGGVTRDIASTDTCFANKHADFDTWFSQRGGSYTNDKACPLIAAVITREVGFVVPGLPGTILRTTNGDFLHFLPRVFSAVGNICYTPSKLIEYTDFATSACVLAAECTIFKDASGKPVPYCYDTNVLEGSVAYESLRPDTRYVLMDGSIIQFPNTYLEGSVRVVTTFDSEYCRHGTCERSEAGVCVSTSGRWVLNNDYYRSLPGVFCGVDAVNLLTNMFTPLIQPIGALDISASIVAGGIVAIVVTCLAYYFMRFRRAFGEYSHVVAFNTLLFLMSFTVLCLTPVYSFLPGVYSVIYLYLTFYLTNDVSFLAHIQWMVMFTPLVPFWITIAYIICISTKHFYWFFSNYLKRRVVFNGVSFSTFEEAALCTFLLNKEMYLKLRSDVLLPLTQYNRYLALYNKYKYFSGAMDTTSYREAACCHLAKALNDFSNSGSDVLYQPPQTSITSAVLQSGFRKMAFPSGKVEGCMVQVTCGTTTLNGLWLDDVVYCPRHVICTSEDMLNPNYEDLLIRKSNHNFLVQAGNVQLRVIGHSMQNCVLKLKVDTANPKTPKYKFVRIQPGQTFSVLACYNGSPSGVYQCAMRPNFTIKGSFLNGSCGSVGFNIDYDCVSFCYMHHMELPTGVHAGTDLEGNFYGPFVDRQTAQAAGTDTTITVNVLAWLYAAVINGDRWFLNRFTTTLNDFNLVAMKYNYEPLTQDHVDILGPLSAQTGIAVLDMCASLKELLQNGMNGRTILGSALLEDEFTPFDVVRQCSGVTFQSAVKRTIKGTHHWLLLTILTSLLVLVQSTQWSLFFFFYENAFLPFAMGIIAMSAFAMMFVKHKHAFLCLFLLPSLATVAYFNMVYMPASWVMRIGSSRKAPYTSTLLPPELSETPNATQPELAPEDPEDSALLEDPVGTVAPQIPPNWHIPSIQDAATPYHPPATPNNMGLIAGAVGGSLLAALVICGIVYWMRRRTQKAPKRIRLPHIREDDQPSSHQPLFY

SEQ ID NO: 105 - DNA, 4326 bp, Artificial Sequence
  Covid19_gD_NSb (DNA) (5′-->3′)
aagcttaccatgatgggaggggctgctgccagactgggagctgtgattctgttcgtggtcattgtggggctgcatggagtgcgggggaaatacgctctggccgatgcaagcctgaaaatggcagaccctaacagattcagagggaaagacctgcccgtgctggatcagctgactgatcccccaggcgtgagacgggtgtatcatatccaagctggcctgccagatccattccagcccccctccctgcctatcaccgtctactacgcagtgctggagagagcctgtaggtccgtgctgctgaatgctcctagcgaagcccctcaaattgtgcggggcgcctccgaagacgtgagaaagcagccatataatctgaccatcgcttggtttaggatgggcggcaactgcgccattcctatcaccgtgatggagtatactgaatgcagctataacaagtccctgggcgcctgcccaattagaacccaacctaggtggaactattatgacagcttttccgcagtcagcgaagacaacctggggttcctgatgcacgcccccgcttttgaaactgccgggacatatctgcggctggtcaaaatcaacgattggacagaaatcacccagttcatcctggaacatagggccaaagggagctgcaaatatgccctgccactgaggatccccccttccgcttgtctgtcccctcaagcctaccagcaaggagtgacagtggacagcatcggcatgctgcccagattcattcccgagaaccagagaactgtggctgtgtactccctgaagattgccggatggcacggatccagcggcgtgccggtggtggatagctattatagcctgctgatgccgattctgaccctgacccgcgcgctgaccgcggaaagccatgtggataccgatctgaccaaaccgtatattaaatgggatctgctgaaatatgattttaccgaagaacgcctgaaactgtttgatcgctattttaaatattgggatcagacctatcatccgaactgcgtgaactgcctggatgatcgctgcattctgcattgcgcgaactttaacgtgctgtttagcaccgtgtttccgccgaccagctttggcccgctggtgcgcaaaatttttgtggatggcgtgccgtttgtggtgagcaccggctatcattttcgcgaactgggcgtggtgcataaccaggatgtgaacctgcatagcagccgcctgagctttaaagaactgctggtgtatgcggcggatccggcgatgcatgcggcgagcggcaacctgctgctggataaacgcaccacctgctttagcgtggcggcgctgaccaacaacgtggcgtttcagaccgtgaaaccgggcaactttaacaaagatttttatgattttgcggtgagcaaaggcttttttaaagaaggcagcagcgtggaactgaaacattttttttttgcgcaggatggcaacgcggcgattagcgattatgattattatcgctataacctgccgaccatgtgcgatattcgccagctgctgtttgtggtggaagtggtggataaatattttgattgctatgatggcggctgcattaacgcgaaccaggtgattgtgaacaacctggataaaagcgcgggctttccgtttaacaaatggggcaaagcgcgcctgtattatgatagcatgagctatgaagatcaggatgcgctgtttgcgtataccaaacgcaacgtgattccgaccattacccagatgaacctgaaatatgcgattagcgcgaaaaaccgcgcgcgcaccgtggcgggcgtgagcatttgcagcaccatgaccaaccgccagtttcatcagaaactgctgaaaagcattgcggcgacccgcggcgcgaccgtggtgattggcaccagcaaattttatggcggctggcataacatgctgaaaaccgtgtatagcgatgtggaaaacccgcatctgatgggctgggattatccgaaatgcgatcgcgcgatgccgaacatgctgcgcattatggcgagcctggtgctggcgcgcaaacataccacctgctgcagcctgagccatcgcttttatcgcctggcgaacgaatgcgcgcaggtgctgagcgaaatggtgatgtgcggcggcagcctgtatgtgaaaccgggcggcaccagcagcggcgatgcgaccaccgcgtatgcgaacagcgtgtttaacatttgccaggcggtgaccgcgaacgtgaacgcgctgctgagcaccgatggcaacaaaattgcggataaatatgtgcgcaacctgcagcatcgcctgtatgaatgcctgtatcgcaaccgcgatgtggataccgattttgtgaacgaattttatgcgtatctgcgcaaacattttagcatgatgattctgagcgatgatgcggtggtgtgctttaacagcacctatgcgagccagggcctggtggcgagcattaaaaactttaaaagcgtgctgtattatcagaacaacgtgtttatgagcgaagcgaaatgctggaccgaaaccgatctgaccaaaggcccgcatgaattttgcagccagcataccatgctggtgaaacagggcgatgattatgtgtatctgccgtatccggatccgagccgcattctgggcgcgggctgctttgtggatgatattgtgaaaaccgatggcaccctgatgattgaacgctttgtgagcctggcgattgatgcgtatccgctgaccaaacatccgaaccaggaatatgcggatgtgtttcatctgtatctgcagtatattcgcaaactgcatgatgaactgaccggccatatgctggatatgtatagcgtgatgctgaccaacgataacaccagccgctattgggaaccggaattttatgaagcgatgtataccccgcataccgtgctgcaggcggtgggcgcgtgcgtgctgtgcaacagccagaccagcctgcgctgcggcgcgtgcattcgccgcccgtttctgtgctgcaaatgctgctatgatcatgtgattagcaccagccataaactggtgctgagcgtgaacccgtatgtgtgcaacgcgccgggctgcgatgtgaccgatgtgacccagctgtatctgggcggcatgagctattattgcaaaagccataaaccgccgattagctttccgctgtgcgcgaacggccaggtgtttggcctgtataaaaacacctgcgtgggcagcgataacgtgaccgattttaacgcgattgcgacctgcgattggaccaacgcgggcgattatattctggcgaacacctgcaccgaacgcctgaaactgtttgcggcggaaaccctgaaagcgaccgaagaaacctttaaactgagctatggcattgcgaccgtgcgcgaagtgctgagcgatcgcgaactgcatctgagctgggaagtgggcaaaccgcgcccgccgctgaaccgcaactatgtgtttaccggctatcgcgtgaccaaaaacagcaaagtgcagattggcgaatatacctttgaaaaaggcgattatggcgatgcggtggtgtatcgcggcaccaccacctataaactgaacgtgggcgattattttgtgctgaccagccataccgtgatgccgctgagcgcgccgaccctggtgccgcaggaacattatgtgcgcattaccggcctgtatccgaccctgaacattagcgatgaatttagcagcaacgtggcgaactatcagaaagtgggcatgcagaaatatagcaccctgcagggcccgccgggcaccggcaaaagccattttgcgattggcctggcgctgtattatccgagcgcgcgcattgtgtataccgcgtgcagccatgcggcggtggatgcgctgtgcgaaaaagcgctgaaatatctgccgattgataaatgcagccgcattggatcctctagaaaggccccttacacttccacactgctgccccctgagctgagcgaaacacccaatgccacccagcccgagctggctcccgaggaccccgaggacagcgccctgctggaggacccagtcggcacagtcgccccacagatccctcccaattggcacattcccagcattcaggacgcagcaaccccctaccacccaccagcaacccctaataatatgggcctgatcgcaggcgcagtcggaggctccctgctggcagcactggtcatctgtgggatcgtctactggatgaggcggcggacacagaaggcaccaaagcggattcggctgccacacattcgggaagacgatcagccttcctcccaccagccactgttttactaactcgag

SEQ ID NO: 106 - protein, 1439 aa, Artificial Sequence
  Covid19_gD_NSb (protein)
KLTMMGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGSSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQAVGACVLCNSQTSLRCGACIRRPFLCCKCCYDHVISTSHKLVLSVNPYVCNAPGCDVTDVTQLYLGGMSYYCKSHKPPISFPLCANGQVFGLYKNTCVGSDNVTDFNAIATCDWTNAGDYILANTCTERLKLFAAETLKATEETFKLSYGIATVREVLSDRELHLSWEVGKPRPPLNRNYVFTGYRVTKNSKVQIGEYTFEKGDYGDAVVYRGTTTYKLNVGDYFVLTSHTVMPLSAPTLVPQEHYVRITGLYPTLNISDEFSSNVANYQKVGMQKYSTLQGPPGTGKSHFAIGLALYYPSARIVYTACSHAAVDALCEKALKYLPIDKCSRIGSSRKAPYTSTLLPPELSETPNATQPELAPEDPEDSALLEDPVGTVAPQIPPNWHIPSIQDAATPYHPPATPNNMGLIAGAVGGSLLAALVICGIVYWMRRRTQKAPKRIRLPHIREDDQPSSHQPLFY

SEQ ID NO: 107 - RNA, 27 nt, Sars-CoV-2
agcgccagcguguacuacagccagcug

SEQ ID NO: 108 - RNA, 24 nt, Sars-CoV-2
guggccgugaagauguucgacgcc

SEQ ID NO: 109 - RNA, 33 nt, Sars-CoV-2
uacgugaacaccuucagcagcaccuucaacgug

SEQ ID NO: 110 - RNA, 45 nt, Sars-CoV-2
uucguggccgccaucuucuaccugaucacccccgugcacgugaug

SEQ ID NO: 111 - RNA, 27 nt, Sars-CoV-2
uucagcgccgugggcaacaucugcuac

SEQ ID NO: 112 - RNA, 27 nt, Sars-CoV-2
gugcccuacugcuacgacaccaacgug

SEQ ID NO: 113 - RNA, 27 nt, Sars-CoV-2
uucaugagguucaggagggccuucggc

SEQ ID NO: 114 - RNA, 54 nt, Sars-CoV-2
caguggauggugauguucaccccccuggugcccuucuggaucaccaucgccuac

SEQ ID NO: 115 - RNA, 27 nt, Sars-CoV-2
agggugguguucaacggcgugagcuuc

SEQ ID NO: 116 - RNA, 27 nt, Sars-CoV-2
gcccugugcaccuuccugcugaacaag

SEQ ID NO: 117 - RNA, 27 nt, Sars-CoV-2
uaccugaagcugaggagcgacgugcug

SEQ ID NO: 118 - RNA, 27 nt, Sars-CoV-2
acccaguacaacagguaccuggcccug

SEQ ID NO: 119 - RNA, 26 nt, Sars-CoV-2
cccccaaguacaaguucgugaggauc

SEQ ID NO: 120 - RNA, 27 nt, Sars-CoV-2
gugagcuucugcuacaugcaccacaug

SEQ ID NO: 121 - RNA, 27 nt, Sars-CoV-2
ggcaccgaccuggagggcaacuucuac

SEQ ID NO: 122 - RNA, 27 nt, Sars-CoV-2
cuggccuggcuguacgccgccgugauc

SEQ ID NO: 123 - RNA, 69 nt, Sars-CoV-2
cugcccuucgccaugggcaucaucgccaugagcgccuucgccaugauguucgugaagcacaagcacgcc

SEQ ID NO: 124 - RNA, 69 nt, Sars-CoV-2
cugcccagccuggccaccguggccuacuucaacaugguguacaugcccgccagcugggugaugaggauc

SEQ ID NO: 125 - RNA, 27 nt, Sars-CoV-2
gugguggacagcuacuacagccugcug

SEQ ID NO: 126 - RNA, 33 nt, Sars-CoV-2
aagaucuucguggacggcgugcccuucguggug

SEQ ID NO: 127 - RNA, 27 nt, Sars-CoV-2
cugguguacgccgccgaccccgccaug

SEQ ID NO: 128 - RNA, 33 nt, Sars-CoV-2
agcguggccgcccugaccaacaacguggccuuc

SEQ ID NO: 129 - RNA, 27 nt, Sars-CoV-2
aucagcgacuacgacuacuacagguac

SEQ ID NO: 130 - RNA, 27 nt, Sars-CoV-2
uucgugguggaggugguggacaaguac

SEQ ID NO: 131 - RNA, 27 nt, Sars-CoV-2
aggcuguacuacgacagcaugagcuac

SEQ ID NO: 132 - RNA, 45 nt, Sars-CoV-2
uucgccuacaccaagaggaacgugauccccaccaucacccagaug

SEQ ID NO: 133 - RNA, 27 nt, Sars-CoV-2
aggcaguuccaccagaagcugcugaag

SEQ ID NO: 134 - RNA, 27 nt, Sars-CoV-2
agggccaugcccaacaugcugaggauc

SEQ ID NO: 135 - RNA, 27 nt, Sars-CoV-2
agccugagccacagguucuacaggcug

SEQ ID NO: 136 - RNA, 57 nt, Sars-CoV-2
gacaccgacuucgugaacgaguucuacgccuaccugaggaagcacuucagcaugaug

SEQ ID NO: 137 - RNA, 27 nt, Sars-CoV-2
cugcccuaccccgaccccagcaggauc

SEQ ID NO: 138 - RNA, 27 nt, Sars-CoV-2
cugaugaucgagagguucgugagccug

SEQ ID NO: 139 - RNA, 27 nt, Sars-CoV-2
caccuguaccugcaguacaucaggaag

SEQ ID NO: 140 - RNA, 27 nt, Sars-CoV-2
cugaccaacgacaacaccagcagguac

SEQ ID NO: 141 - RNA, 27 nt, Sars-CoV-2
gugaccgacgugacccagcuguaccug

SEQ ID NO: 142 - RNA, 27 nt, Sars-CoV-2
augagcuacuacugcaagagccacaag

SEQ ID NO: 143 - RNA, 27 nt, Sars-CoV-2
uucccccugugcgccaacggccaggug

SEQ ID NO: 144 - RNA, 27 nt, Sars-CoV-2
aagcuguucgccgccgagacccugaag

SEQ ID NO: 145 - RNA, 27 nt, Sars-CoV-2
gagaccuucaagcugagcuacggcauc

SEQ ID NO: 146 - RNA, 27 nt, Sars-CoV-2
aaggugcagaucggcgaguacaccuuc

SEQ ID NO: 147 - RNA, 27 nt, Sars-CoV-2
gugguguacaggggcaccaccaccuac

SEQ ID NO: 148 - RNA, 27 nt, Sars-CoV-2
augccccugagcgcccccacccuggug

SEQ ID NO: 149 - RNA, 27 nt, Sars-CoV-2
gugggcaugcagaaguacagcacccug

SEQ ID NO: 150 - RNA, 27 nt, Sars-CoV-2
uucgccaucggccuggcccuguacuac

SEQ ID NO: 151 - RNA, 27 nt, Sars-CoV-2
uacaccgccugcagccacgccgccgug

SEQ ID NO: 152 - RNA, 27 nt, Sars-CoV-2
cugcccaucgacaagugcagcaggauc

SEQ ID NO: 153 - RNA, 12 nt, Artificial Sequence
  Spacer RNA Sequence
ggcggcggcagc

SEQ ID NO: 154 - RNA, 1974 nt, Artificial Sequence
  Polyepitope RNA sequence
agcgccagcguguacuacagccagcugggcggcggcagcguggccgugaagauguucgacgccggcggcggcagcuacgugaacaccuucagcagcaccuucaacgugggcggcggcagcuucguggccgccaucuucuaccugaucacccccgugcacgugaugggcggcggcagcuucagcgccgugggcaacaucugcuacggcggcggcagcgugcccuacugcuacgacaccaacgugggcggcggcagcuucaugagguucaggagggccuucggcggcggcggcagccaguggauggugauguucaccccccuggugcccuucuggaucaccaucgccuacggcggcggcagcagggugguguucaacggcgugagcuucggcggcggcagcgcccugugcaccuuccugcugaacaagggcggcggcagcuaccugaagcugaggagcgacgugcugggcggcggcagcacccaguacaacagguaccuggcccugggcggcggcagcacccccaaguacaaguucgugaggaucggcggcggcagcgugagcuucugcuacaugcaccacaugggcggcggcagcggcaccgaccuggagggcaacuucuacggcggcggcagccuggccuggcuguacgccgccgugaucggcggcggcagccugcccuucgccaugggcaucaucgccaugagcgccuucgccaugauguucgugaagcacaagcacgccggcggcggcagccugcccagccuggccaccguggccuacuucaacaugguguacaugcccgccagcugggugaugaggaucggcggcggcagcgugguggacagcuacuacagccugcugggcggcggcagcaagaucuucguggacggcgugcccuucguggugggcggcggcagccugguguacgccgccgaccccgccaugggcggcggcagcagcguggccgcccugaccaacaacguggccuucggcggcggcagcaucagcgacuacgacuacuacagguacggcggcggcagcuucgugguggaggugguggacaaguacggcggcggcagcaggcuguacuacgacagcaugagcuacggcggcggcagcuucgccuacaccaagaggaacgugauccccaccaucacccagaugggcggcggcagcaggcaguuccaccagaagcugcugaagggcggcggcagcagggccaugcccaacaugcugaggaucggcggcggcagcagccugagccacagguucuacaggcugggcggcggcagcgacaccgacuucgugaacgaguucuacgccuaccugaggaagcacuucagcaugaugggcggcggcagccugcccuaccccgaccccagcaggaucggcggcggcagccugaugaucgagagguucgugagccugggcggcggcagccaccuguaccugcaguacaucaggaagggcggcggcagccugaccaacgacaacaccagcagguacggcggcggcagcgugaccgacgugacccagcuguaccugggcggcggcagcaugagcuacuacugcaagagccacaagggcggcggcagcuucccccugugcgccaacggccaggugggcggcggcagcaagcuguucgccgccgagacccugaagggcggcggcagcgagaccuucaagcugagcuacggcaucggcggcggcagcaaggugcagaucggcgaguacaccuucggcggcggcagcgugguguacaggggcaccaccaccuacggcggcggcagcaugccccugagcgcccccacccuggugggcggcggcagcgugggcaugcagaaguacagcacccugggcggcggcagcuucgccaucggccuggcccuguacuacggcggcggcagcuacaccgccugcagccacgccgccgugggcggcggcagccugcccaucgacaagugcagcaggauc

SEQ ID NO: 155 - RNA, 819 nt, Artificial Sequence
  RNA Sequence of the glycoprotein D (gD) n-terminal fragment
aagcuuaccaugaugggaggggcugcugccagacugggagcugugauucuguucguggucauuguggggcugcauggagugcgggggaaauacgcucuggccgaugcaagccugaaaauggcagacccuaacagauucagagggaaagaccugcccgugcuggaucagcugacugaucccccaggcgugagacggguguaucauauccaagcuggccugccagauccauuccagccccccucccugccuaucaccgucuacuacgcagugcuggagagagccuguagguccgugcugcugaaugcuccuagcgaagccccucaaauugugcggggcgccuccgaagacgugagaaagcagccauauaaucugaccaucgcuugguuuaggaugggcggcaacugcgccauuccuaucaccgugauggaguauacugaaugcagcuauaacaagucccugggcgccugcccaauuagaacccaaccuagguggaacuauuaugacagcuuuuccgcagucagcgaagacaaccugggguuccugaugcacgcccccgcuuuugaaacugccgggacauaucugcggcuggucaaaaucaacgauuggacagaaaucacccaguucauccuggaacauagggccaaagggagcugcaaauaugcccugccacugaggauccccccuuccgcuugucuguccccucaagccuaccagcaaggagugacaguggacagcaucggcaugcugcccagauucauucccgagaaccagagaacuguggcuguguacucccugaagauugccggauggcacggaucc

SEQ ID NO: 156 - RNA, 396 nt, Artificial Sequence
  RNA Sequence of the gD C-terminal fragment
ggauccucuagaaaggccccuuacacuuccacacugcugcccccugagcugagcgaaacacccaaugccacccagcccgagcuggcucccgaggaccccgaggacagcgcccugcuggaggacccagucggcacagucgccccacagaucccucccaauuggcacauucccagcauucaggacgcagcaacccccuaccacccaccagcaaccccuaauaauaugggccugaucgcaggcgcagucggaggcucccugcuggcagcacuggucaucugugggaucgucuacuggaugaggcggcggacacagaaggcaccaaagcggauucggcugccacacauucgggaagacgaucagccuuccucccaccagccacuguuuuacuaacucgag

SEQ ID NO: 157 - RNA, 3189 nt, Artificial Sequence
  Covid19_gD_POLIEP (RNA) (5′-->3′)
aagcuuaccaugaugggaggggcugcugccagacugggagcugugauucuguucguggucauuguggggcugcauggagugcgggggaaauacgcucuggccgaugcaagccugaaaauggcagacccuaacagauucagagggaaagaccugcccgugcuggaucagcugacugaucccccaggcgugagacggguguaucauauccaagcuggccugccagauccauuccagccccccucccugccuaucaccgucuacuacgcagugcuggagagagccuguagguccgugcugcugaaugcuccuagcgaagccccucaaauugugcggggcgccuccgaagacgugagaaagcagccauauaaucugaccaucgcuugguuuaggaugggcggcaacugcgccauuccuaucaccgugauggaguauacugaaugcagcuauaacaagucccugggcgccugcccaauuagaacccaaccuagguggaacuauuaugacagcuuuuccgcagucagcgaagacaaccugggguuccugaugcacgcccccgcuuuugaaacugccgggacauaucugcggcuggucaaaaucaacgauuggacagaaaucacccaguucauccuggaacauagggccaaagggagcugcaaauaugcccugccacugaggauccccccuuccgcuugucuguccccucaagccuaccagcaaggagugacaguggacagcaucggcaugcugcccagauucauucccgagaaccagagaacuguggcuguguacucccugaagauugccggauggcacggauccagcgccagcguguacuacagccagcugggcggcggcagcguggccgugaagauguucgacgccggcggcggcagcuacgugaacaccuucagcagcaccuucaacgugggcggcggcagcuucguggccgccaucuucuaccugaucacccccgugcacgugaugggcggcggcagcuucagcgccgugggcaacaucugcuacggcggcggcagcgugcccuacugcuacgacaccaacgugggcggcggcagcuucaugagguucaggagggccuucggcggcggcggcagccaguggauggugauguucaccccccuggugcccuucuggaucaccaucgccuacggcggcggcagcagggugguguucaacggcgugagcuucggcggcggcagcgcccugugcaccuuccugcugaacaagggcggcggcagcuaccugaagcugaggagcgacgugcugggcggcggcagcacccaguacaacagguaccuggcccugggcggcggcagcacccccaaguacaaguucgugaggaucggcggcggcagcgugagcuucugcuacaugcaccacaugggcggcggcagcggcaccgaccuggagggcaacuucuacggcggcggcagccuggccuggcuguacgccgccgugaucggcggcggcagccugcccuucgccaugggcaucaucgccaugagcgccuucgccaugauguucgugaagcacaagcacgccggcggcggcagccugcccagccuggccaccguggccuacuucaacaugguguacaugcccgccagcugggugaugaggaucggcggcggcagcgugguggacagcuacuacagccugcugggcggcggcagcaagaucuucguggacggcgugcccuucguggugggcggcggcagccugguguacgccgccgaccccgccaugggcggcggcagcagcguggccgcccugaccaacaacguggccuucggcggcggcagcaucagcgacuacgacuacuacagguacggcggcggcagcuucgugguggaggugguggacaaguacggcggcggcagcaggcuguacuacgacagcaugagcuacggcggcggcagcuucgccuacaccaagaggaacgugauccccaccaucacccagaugggcggcggcagcaggcaguuccaccagaagcugcugaagggcggcggcagcagggccaugcccaacaugcugaggaucggcggcggcagcagccugagccacagguucuacaggcugggcggcggcagcgacaccgacuucgugaacgaguucuacgccuaccugaggaagcacuucagcaugaugggcggcggcagccugcccuaccccgaccccagcaggaucggcggcggcagccugaugaucgagagguucgugagccugggcggcggcagccaccuguaccugcaguacaucaggaagggcggcggcagccugaccaacgacaacaccagcagguacggcggcggcagcgugaccgacgugacccagcuguaccugggcggcggcagcaugagcuacuacugcaagagccacaagggcggcggcagcuucccccugugcgccaacggccaggugggcggcggcagcaagcuguucgccgccgagacccugaagggcggcggcagcgagaccuucaagcugagcuacggcaucggcggcggcagcaaggugcagaucggcgaguacaccuucggcggcggcagcgugguguacaggggcaccaccaccuacggcggcggcagcaugccccugagcgcccccacccuggugggcggcggcagcgugggcaugcagaaguacagcacccugggcggcggcagcuucgccaucggccuggcccuguacuacggcggcggcagcuacaccgccugcagccacgccgccgugggcggcggcagccugcccaucgacaagugcagcaggaucggauccucuagaaaggccccuuacacuuccacacugcugcccccugagcugagcgaaacacccaaugccacccagcccgagcuggcucccgaggaccccgaggacagcgcccugcuggaggacccagucggcacagucgccccacagaucccucccaauuggcacauucccagcauucaggacgcagcaacccccuaccacccaccagcaaccccuaauaauaugggccugaucgcaggcgcagucggaggcucccugcuggcagcacuggucaucugugggaucgucuacuggaugaggcggcggacacagaaggcaccaaagcggauucggcugccacacauucgggaagacgaucagccuuccucccaccagccacuguuuuacuaacucgag

SEQ ID NO: 158 - RNA, 4548 nt, Artificial Sequence
  Covid19_gD_NSa (RNA) (5′-->3′)
aagcuuaccaugaugggaggggcugcugccagacugggagcugugauucuguucguggucauuguggggcugcauggagugcgggggaaauacgcucuggccgaugcaagccugaaaauggcagacccuaacagauucagagggaaagaccugcccgugcuggaucagcugacugaucccccaggcgugagacggguguaucauauccaagcuggccugccagauccauuccagccccccucccugccuaucaccgucuacuacgcagugcuggagagagccuguagguccgugcugcugaaugcuccuagcgaagccccucaaauugugcggggcgccuccgaagacgugagaaagcagccauauaaucugaccaucgcuugguuuaggaugggcggcaacugcgccauuccuaucaccgugauggaguauacugaaugcagcuauaacaagucccugggcgccugcccaauuagaacccaaccuagguggaacuauuaugacagcuuuuccgcagucagcgaagacaaccugggguuccugaugcacgcccccgcuuuugaaacugccgggacauaucugcggcuggucaaaaucaacgauuggacagaaaucacccaguucauccuggaacauagggccaaagggagcugcaaauaugcccugccacugaggauccccccuuccgcuugucuguccccucaagccuaccagcaaggagugacaguggacagcaucggcaugcugcccagauucauucccgagaaccagagaacuguggcuguguacucccugaagauugccggauggcacggauccagcgcgaaaagcgcgagcguguauuauagccagcugaugugccagccgauucugcugcuggaucaggcgcuggugagcgaugugggcgauagcgcggaaguggcggugaaaauguuugaugcguaugugaacaccuuuagcagcaccuuuaacgugccgauggaaaaacugaaaacccugguggcgaccgcggaagcggaacuggcgaaaaacgugagccuggauaacgugcugagcaccuuuauuagcgcggcgcgccagggcuuuguggauagcgauguggaaaccaaagaugugguggaaugccugaaacugagccaucagagcgauauugaagugaccggcgauagcugcaacaacuauaugcugaccuauaacaaaguggaaaacaugaccccgcgcgaucugggcgcgugcauugauugcagcgcgcgccauauuaacgcgcagguggcgaaaagccauaacauugcgcugauuuggaacgugaaagauuuuaugagccugagcgaacagcugcgcaaacagauucgcagcgcggcgaaaaaaaacaaccugccguuuaaacugaccugcgcgaccacccgccagguggugaacguggugaccaccaaaauugcgcugaaaggcggcaaaauugugaacaacuggcugaaacagcugauuaaagugacccugguguuucuguuuguggcggcgauuuuuuaucugauuaccccggugcaugugaugagcaaacauaccgauuuuagcagcgaaauuauuggcuauaaagcgauugauggcggcgugacccgcgauauugcgagcaccgauaccugcuuugcgaacaaacaugcggauuuugauaccugguuuagccagcgcggcggcagcuauaccaacgauaaagcgugcccgcugauugcggcggugauuacccgcgaagugggcuuuguggugccgggccugccgggcaccauucugcgcaccaccaacggcgauuuucugcauuuucugccgcgcguguuuagcgcggugggcaacauuugcuauaccccgagcaaacugauugaauauaccgauuuugcgaccagcgcgugcgugcuggcggcggaaugcaccauuuuuaaagaugcgagcggcaaaccggugccguauugcuaugauaccaacgugcuggaaggcagcguggcguaugaaagccugcgcccggauacccgcuaugugcugauggauggcagcauuauucaguuuccgaacaccuaucuggaaggcagcgugcgcguggugaccaccuuugauagcgaauauugccgccauggcaccugcgaacgcagcgaagcgggcgugugcgugagcaccagcggccgcugggugcugaacaacgauuauuaucgcagccugccgggcguguuuugcggcguggaugcggugaaccugcugaccaacauguuuaccccgcugauucagccgauuggcgcgcuggauauuagcgcgagcauuguggcgggcggcauuguggcgauuguggugaccugccuggcguauuauuuuaugcgcuuucgccgcgcguuuggcgaauauagccaugugguggcguuuaacacccugcuguuucugaugagcuuuaccgugcugugccugaccccgguguauagcuuucugccgggcguguauagcgugauuuaucuguaucugaccuuuuaucugaccaacgaugugagcuuucuggcgcauauucaguggauggugauguuuaccccgcuggugccguuuuggauuaccauugcguauauuauuugcauuagcaccaaacauuuuuauugguuuuuuagcaacuaucugaaacgccgcgugguguuuaacggcgugagcuuuagcaccuuugaagaagcggcgcugugcaccuuucugcugaacaaagaaauguaucugaaacugcgcagcgaugugcugcugccgcugacccaguauaaccgcuaucuggcgcuguauaacaaauauaaauauuuuagcggcgcgauggauaccaccagcuaucgcgaagcggcgugcugccaucuggcgaaagcgcugaacgauuuuagcaacagcggcagcgaugugcuguaucagccgccgcagaccagcauuaccagcgcggugcugcagagcggcuuucgcaaaauggcguuuccgagcggcaaaguggaaggcugcauggugcaggugaccugcggcaccaccacccugaacggccuguggcuggaugaugugguguauugcccgcgccaugugauuugcaccagcgaagauaugcugaacccgaacuaugaagaucugcugauucgcaaaagcaaccauaacuuucuggugcaggcgggcaacgugcagcugcgcgugauuggccauagcaugcagaacugcgugcugaaacugaaaguggauaccgcgaacccgaaaaccccgaaauauaaauuugugcgcauucagccgggccagaccuuuagcgugcuggcgugcuauaacggcagcccgagcggcguguaucagugcgcgaugcgcccgaacuuuaccauuaaaggcagcuuucugaacggcagcugcggcagcgugggcuuuaacauugauuaugauugcgugagcuuuugcuauaugcaucauauggaacugccgaccggcgugcaugcgggcaccgaucuggaaggcaacuuuuauggcccguuuguggaucgccagaccgcgcaggcggcgggcaccgauaccaccauuaccgugaacgugcuggcguggcuguaugcggcggugauuaacggcgaucgcugguuucugaaccgcuuuaccaccacccugaacgauuuuaaccugguggcgaugaaauauaacuaugaaccgcugacccaggaucauguggauauucugggcccgcugagcgcgcagaccggcauugcggugcuggauaugugcgcgagccugaaagaacugcugcagaacggcaugaacggccgcaccauucugggcagcgcgcugcuggaagaugaauuuaccccguuugauguggugcgccagugcagcggcgugaccuuucagagcgcggugaaacgcaccauuaaaggcacccaucauuggcugcugcugaccauucugaccagccugcuggugcuggugcagagcacccaguggagccuguuuuuuuuuuuuuaugaaaacgcguuucugccguuugcgaugggcauuauugcgaugagcgcguuugcgaugauguuugugaaacauaaacaugcguuucugugccuguuucugcugccgagccuggcgaccguggcguauuuuaacaugguguauaugccggcgagcugggugaugcgcauuggauccucuagaaaggccccuuacacuuccacacugcugcccccugagcugagcgaaacacccaaugccacccagcccgagcuggcucccgaggaccccgaggacagcgcccugcuggaggacccagucggcacagucgccccacagaucccucccaauuggcacauucccagcauucaggacgcagcaacccccuaccacccaccagcaaccccuaauaauaugggccugaucgcaggcgcagucggaggcucccugcuggcagcacuggucaucugugggaucgucuacuggaugaggcggcggacacagaaggcaccaaagcggauucggcugccacacauucgggaagacgaucagccuuccucccaccagccacuguuuuacuaacucgag

SEQ ID NO: 159 - RNA, 4326 nt, Artificial Sequence
  Covid19_gD_NSb (RNA) (5′-->3′)
aagcuuaccaugaugggaggggcugcugccagacugggagcugugauucuguucguggucauuguggggcugcauggagugcgggggaaauacgcucuggccgaugcaagccugaaaauggcagacccuaacagauucagagggaaagaccugcccgugcuggaucagcugacugaucccccaggcgugagacggguguaucauauccaagcuggccugccagauccauuccagccccccucccugccuaucaccgucuacuacgcagugcuggagagagccuguagguccgugcugcugaaugcuccuagcgaagccccucaaauugugcggggcgccuccgaagacgugagaaagcagccauauaaucugaccaucgcuugguuuaggaugggcggcaacugcgccauuccuaucaccgugauggaguauacugaaugcagcuauaacaagucccugggcgccugcccaauuagaacccaaccuagguggaacuauuaugacagcuuuuccgcagucagcgaagacaaccugggguuccugaugcacgcccccgcuuuugaaacugccgggacauaucugcggcuggucaaaaucaacgauuggacagaaaucacccaguucauccuggaacauagggccaaagggagcugcaaauaugcccugccacugaggauccccccuuccgcuugucuguccccucaagccuaccagcaaggagugacaguggacagcaucggcaugcugcccagauucauucccgagaaccagagaacuguggcuguguacucccugaagauugccggauggcacggauccagcggcgugccggugguggauagcuauuauagccugcugaugccgauucugacccugacccgcgcgcugaccgcggaaagccauguggauaccgaucugaccaaaccguauauuaaaugggaucugcugaaauaugauuuuaccgaagaacgccugaaacuguuugaucgcuauuuuaaauauugggaucagaccuaucauccgaacugcgugaacugccuggaugaucgcugcauucugcauugcgcgaacuuuaacgugcuguuuagcaccguguuuccgccgaccagcuuuggcccgcuggugcgcaaaauuuuuguggauggcgugccguuuguggugagcaccggcuaucauuuucgcgaacugggcguggugcauaaccaggaugugaaccugcauagcagccgccugagcuuuaaagaacugcugguguaugcggcggauccggcgaugcaugcggcgagcggcaaccugcugcuggauaaacgcaccaccugcuuuagcguggcggcgcugaccaacaacguggcguuucagaccgugaaaccgggcaacuuuaacaaagauuuuuaugauuuugcggugagcaaaggcuuuuuuaaagaaggcagcagcguggaacugaaacauuuuuuuuuugcgcaggauggcaacgcggcgauuagcgauuaugauuauuaucgcuauaaccugccgaccaugugcgauauucgccagcugcuguuugugguggaagugguggauaaauauuuugauugcuaugauggcggcugcauuaacgcgaaccaggugauugugaacaaccuggauaaaagcgcgggcuuuccguuuaacaaauggggcaaagcgcgccuguauuaugauagcaugagcuaugaagaucaggaugcgcuguuugcguauaccaaacgcaacgugauuccgaccauuacccagaugaaccugaaauaugcgauuagcgcgaaaaaccgcgcgcgcaccguggcgggcgugagcauuugcagcaccaugaccaaccgccaguuucaucagaaacugcugaaaagcauugcggcgacccgcggcgcgaccguggugauuggcaccagcaaauuuuauggcggcuggcauaacaugcugaaaaccguguauagcgauguggaaaacccgcaucugaugggcugggauuauccgaaaugcgaucgcgcgaugccgaacaugcugcgcauuauggcgagccuggugcuggcgcgcaaacauaccaccugcugcagccugagccaucgcuuuuaucgccuggcgaacgaaugcgcgcaggugcugagcgaaauggugaugugcggcggcagccuguaugugaaaccgggcggcaccagcagcggcgaugcgaccaccgcguaugcgaacagcguguuuaacauuugccaggcggugaccgcgaacgugaacgcgcugcugagcaccgauggcaacaaaauugcggauaaauaugugcgcaaccugcagcaucgccuguaugaaugccuguaucgcaaccgcgauguggauaccgauuuugugaacgaauuuuaugcguaucugcgcaaacauuuuagcaugaugauucugagcgaugaugcgguggugugcuuuaacagcaccuaugcgagccagggccugguggcgagcauuaaaaacuuuaaaagcgugcuguauuaucagaacaacguguuuaugagcgaagcgaaaugcuggaccgaaaccgaucugaccaaaggcccgcaugaauuuugcagccagcauaccaugcuggugaaacagggcgaugauuauguguaucugccguauccggauccgagccgcauucugggcgcgggcugcuuuguggaugauauugugaaaaccgauggcacccugaugauugaacgcuuugugagccuggcgauugaugcguauccgcugaccaaacauccgaaccaggaauaugcggauguguuucaucuguaucugcaguauauucgcaaacugcaugaugaacugaccggccauaugcuggauauguauagcgugaugcugaccaacgauaacaccagccgcuauugggaaccggaauuuuaugaagcgauguauaccccgcauaccgugcugcaggcggugggcgcgugcgugcugugcaacagccagaccagccugcgcugcggcgcgugcauucgccgcccguuucugugcugcaaaugcugcuaugaucaugugauuagcaccagccauaaacuggugcugagcgugaacccguaugugugcaacgcgccgggcugcgaugugaccgaugugacccagcuguaucugggcggcaugagcuauuauugcaaaagccauaaaccgccgauuagcuuuccgcugugcgcgaacggccagguguuuggccuguauaaaaacaccugcgugggcagcgauaacgugaccgauuuuaacgcgauugcgaccugcgauuggaccaacgcgggcgauuauauucuggcgaacaccugcaccgaacgccugaaacuguuugcggcggaaacccugaaagcgaccgaagaaaccuuuaaacugagcuauggcauugcgaccgugcgcgaagugcugagcgaucgcgaacugcaucugagcugggaagugggcaaaccgcgcccgccgcugaaccgcaacuauguguuuaccggcuaucgcgugaccaaaaacagcaaagugcagauuggcgaauauaccuuugaaaaaggcgauuauggcgaugcggugguguaucgcggcaccaccaccuauaaacugaacgugggcgauuauuuugugcugaccagccauaccgugaugccgcugagcgcgccgacccuggugccgcaggaacauuaugugcgcauuaccggccuguauccgacccugaacauuagcgaugaauuuagcagcaacguggcgaacuaucagaaagugggcaugcagaaauauagcacccugcagggcccgccgggcaccggcaaaagccauuuugcgauuggccuggcgcuguauuauccgagcgcgcgcauuguguauaccgcgugcagccaugcggcgguggaugcgcugugcgaaaaagcgcugaaauaucugccgauugauaaaugcagccgcauuggauccucuagaaaggccccuuacacuuccacacugcugcccccugagcugagcgaaacacccaaugccacccagcccgagcuggcucccgaggaccccgaggacagcgcccugcuggaggacccagucggcacagucgccccacagaucccucccaauuggcacauucccagcauucaggacgcagcaacccccuaccacccaccagcaaccccuaauaauaugggccugaucgcaggcgcagucggaggcucccugcuggcagcacuggucaucugugggaucgucuacuggaugaggcggcggacacagaaggcaccaaagcggauucggcugccacacauucgggaagacgaucagccuuccucccaccagccacuguuuuacuaacucgag

SEQ ID NO: 160 - protein, 9 aa, Sars-CoV-2
VAYFNMVYM